(19) United States
(12) Patent Application Publication
(10) Pub. No.: US 2019/0307784 A1
(43) Pub. Date: Oct. 10, 2019

(54) APTAMERS REDUCE SICKLE HEMOGLOBIN POLYMERIZATION

(71) Applicant: THE JOHNS HOPKINS UNIVERSITY, Baltimore, MD (US)

(72) Inventors: James F. Casella, Baltimore, MD (US); Emily Barron-Casella, Baltimore, MD (US); Jeffrey R. Keefer, Baltimore, MD (US); Yolanda Fortenberry, Pikesville, MD (US); Shirley H. Purvis, Baltimore, MD (US)

(21) Appl. No.: 16/089,036

(22) PCT Filed: Mar. 30, 2017
(86) PCT No.: PCT/US2017/024918

§ 371 Date: Sep. 27, 2018

Related U.S. Application Data
(60) Provisional application No. 62/315,942, filed on Mar. 31, 2016.

(51) Int. Cl. A61K 31/7088; A61P 7/06; C12N 15/115

(52) U.S. Cl. CPC A61K 31/7088; C12N 15/115; A61P 7/06

(57) ABSTRACT

The presently disclosed subject matter provides methods for reducing sickling of an erythrocyte comprising sickle hemoglobin (HbS) by introducing polynucleotide aptamers into the erythrocyte. The polynucleotide aptamers specifically bind to HbS to inhibit polymerization of the HbS without affecting the oxygen affinity of the HbS.

### REFERENCE TO RELATED APPLICATIONS

This application is a 35 U.S.C. § 371 U.S. national entry of International Application PCT/US2017/024918, having an international filing date of Mar. 30, 2017, which claims the benefit of U.S. Provisional Application No. 62/315,942, filed Mar. 31, 2016, the content of each of the aforementioned applications is herein incorporated by reference in their entirety.

### INCORPORATION-BY-REFERENCE OF MATERIAL SUBMITTED ELECTRONICALLY

This application contains a sequence listing. It has been submitted electronically via EFS-Web as an ASCII text file entitled “111232-00495_ST25.txt”. The sequence listing is 20,480 bytes in size, and was created on Mar. 8, 2016. It is hereby incorporated by reference in its entirety.

### BACKGROUND OF THE INVENTION

Sickle cell disease (SCD) results from the single amino acid substitution of valine for glutamic acid at the 136 position of sickle hemoglobin, causing hemoglobin monomers to polymerize upon deoxygenation in the microvasculature (Serjeant, 1985). Deoxygenation involves a transition from a relaxed, or R-state, conformation to a tense, or T-state, conformation (Perutz et al, 1998). It is in this deoxygenated conformation that sickle hemoglobin (HbS) aggregates to form 14-strand polymers (Dykes et al, 1978), through a two-step mechanism called “double-nucleation.” In the first step, homogeneous nucleation, molecules randomly assemble until reaching a critical nucleus that is large enough to be stable. Once stable nuclei occur, polymerization is thermodynamically favorable and fibers begin to elongate (Ferrone et al, 1985). The time required for stable nuclei to form following deoxygenation is known as the delay time, and is highly dependent on hemoglobin concentration (Hofrichter et al, 1974). In the second step, heterogeneous nucleation, nucleation takes place directly on the stable fiber, with secondary fibers presenting further nucleation sites, so that growth in this phase becomes exponential (Hofrichter, 1986). This polymerization leads to rigid and abnormally sickle-shaped cells that can occlude capillaries and venules, resulting in pain, organ damage, susceptibility to infection and early death (Steinberg et al, 1999).

Current therapies shown to alter the severity of the disease, such as bone marrow transplantation and blood transfusion, are hindered by complications and limitations (King & Shenoy, 2015; Yazdanbakhsh et al., 2012). Hydroxyurea, a drug that ameliorates symptoms of SCD by increasing the production of fetal hemoglobin (HbF), which inhibits the polymerization phase (Nagel et al, 1979), may be associated with adverse side effects and is not an effective therapy for all patients (Ataga & Stocker, 2015).

### SUMMARY OF THE INVENTION

The practice of the presently disclosed subject matter will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and

Sambrook, Molecular Cloning. A Laboratory Manual, 3rd ed., Cold Spring Harbor Laboratory Press, Cold Spring Harbor, 2001; Harlow, E. and Lane, D., Antibodies-A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: www.ncbi.nlm.nih.gov/omim/ and in Online Mendelian Inheritance in Animals (OMIA), a database of genes, inherited disorders and traits in animal species (other than human and mouse), at omia.angis.org.au/contact.shtml.

In some aspects, the presently disclosed subject matter provides a method for inhibiting sickling of an erythrocyte, the method comprising introducing at least one polynucleotide aptamer into an erythrocyte comprising at least a first sickle hemoglobin (HbS) and a second HbS under conditions effective to specifically bind the at least one polynucleotide aptamer to the first HbS, wherein specifically binding the at least one polynucleotide aptamer to the first HbS inhibits polymerization of the first HbS with the second HbS, thereby inhibiting sickling of the erythrocyte.

In some embodiments, specifically binding at least one polynucleotide aptamer to the first HbS inhibits polymerization of the first HbS with a second HbS without affecting the oxygen affinity of the first HbS. In some embodiments, specifically binding at least one polynucleotide aptamer to the first HbS occurs under physiological conditions. In some embodiments, specifically binding at least one polynucleotide aptamer to the first HbS occurs under hypoxic conditions. In some embodiments, at least one polynucleotide aptamer is an RNA aptamer.

In some embodiments, the first HbS and/or the second HbS is a monomer. In some embodiments, the first HbS and/or the second HbS is a polymer.

In some embodiments, specifically binding at least one polynucleotide aptamer to the first HbS increases the delay time before polymerization of the first HbS with the second HbS occurs. In some embodiments, specifically binding at least one polynucleotide aptamer to the first HbS increases the delay time before polymerization of the first HbS with the second HbS occurs by at least about 2-fold.

In some embodiments, specifically binding at least one polynucleotide aptamer to the first HbS reduces the rate of polymerization of the first HbS with the second HbS. In some embodiments, specifically binding at least one polynucleotide aptamer to the first HbS reduces the rate of polymerization of the first HbS with the second HbS by at least about 60%.

In some embodiments, at least one polynucleotide aptamer specifically binds oxygenated HbS. In some embodiments, at least one polynucleotide aptamer specifically binds deoxygenated HbS. In some embodiments, at least one polynucleotide aptamer specifically binds both oxygenated HbS and deoxygenated HbS. In some embodiments, at least one polynucleotide aptamer specifically binds both oxygenated HbS and deoxygenated HbS with similar affinity.

In some embodiments, specifically binding at least one polynucleotide aptamer to the first HbS reduces the rate and extent of polymerization of the first HbS with the second HbS. In some embodiments, at least one polynucleotide aptamer comprises a nucleotide sequence selected from the group consisting of: (a) a nucleotide sequence at least 80% identical to SEQ ID NO: 1; (b) a nucleotide sequence at least 90% identical to SEQ ID NO: 1; (c) a nucleotide sequence at least 95% identical to SEQ ID NO: 1; (d) a nucleotide sequence at least 99% identical to SEQ ID NO: 1; (e) the nucleotide sequence of SEQ ID NO: 1; (f) a nucleotide sequence at least 80% identical to SEQ ID NO: 9; (g) a nucleotide sequence at least 90% identical to SEQ ID NO: 9; (h) a nucleotide sequence at least 95% identical to SEQ ID NO: 9; (i) a nucleotide sequence at least 99% identical to SEQ ID NO: 9; and (j) the nucleotide sequence of SEQ ID NO: 9.

In some embodiments, specifically binding at least one polynucleotide aptamer to the first HbS reduces the rate of polymerization without reducing the extent of polymerization of the first HbS with the second HbS. In some embodiments, at least one polynucleotide aptamer comprises a nucleotide sequence selected from the group consisting of: (a) a nucleotide sequence at least 80% identical to SEQ ID NO: 2; (b) a nucleotide sequence at least 90% identical to SEQ ID NO: 2; (c) a nucleotide sequence at least 95% identical to SEQ ID NO: 2; (d) a nucleotide sequence at least 99% identical to SEQ ID NO: 2; (e) the nucleotide sequence of SEQ ID NO: 2; (f) a nucleotide sequence at least 80% identical to SEQ ID NO: 43; (g) a nucleotide sequence at least 90% identical to SEQ ID NO: 43; (h) a nucleotide sequence at least 95% identical to SEQ ID NO: 43; (i) a nucleotide sequence at least 99% identical to SEQ ID NO: 43; and (j) the nucleotide sequence of SEQ ID NO: 43.

In some embodiments, at least one polynucleotide aptamer is modified to prevent nuclease degradation. In some embodiments, at least one polynucleotide aptamer comprises at least one 2′-fluoro nucleotide.

In certain aspects, the presently disclosed subject matter provides a method for treating or preventing sickle cell disease in a subject in need thereof, the method comprising administering to a subject a therapeutically effective amount of a polynucleotide aptamer, wherein the polynucleotide aptamer specifically binds to a first HbS in an erythrocyte in the subject, wherein specifically binding the polynucleotide aptamer to the first HbS inhibits polymerization of the first HbS with a second HbS in the erythrocyte, thereby inhibiting sickling of the erythrocyte and treating or preventing sickle cell disease in the subject. In some embodiments, the polynucleotide aptamer is delivered into the erythrocyte.

In some embodiments, the polynucleotide aptamer comprises a nucleotide sequence selected from the group consisting of: (a) a nucleotide sequence at least 80% identical to SEQ ID NO: 1; (b) a nucleotide sequence at least 90% identical to SEQ ID NO: 1; (c) a nucleotide sequence at least 95% identical to SEQ ID NO: 1; (d) a nucleotide sequence at least 99% identical to SEQ ID NO: 1; (e) the nucleotide sequence of SEQ ID NO: 1; (f) a nucleotide sequence at least 80% identical to SEQ ID NO: 9; (g) a nucleotide sequence at least 90% identical to SEQ ID NO:9; (h) a nucleotide sequence at least 95% identical to SEQ ID NO:9; (i) a nucleotide sequence at least 99% identical to SE ID NO: 9; (j) the nucleotide sequence of SEQ ID NO: 9; (k) a nucleotide sequence at least 80% identical to SEQ ID NO: 2; (1) a nucleotide sequence at least 90% identical to SEQ ID NO: 2; (m) a nucleotide sequence at least 95% identical to SEQ ID NO: 2; (n) a nucleotide sequence at least 99% identical to SEQ ID NO: 2; (o) the nucleotide sequence of SEQ ID NO: 2; (p) a nucleotide sequence at least 80% identical to SEQ ID NO: 43; (q) a nucleotide sequence at least 90% identical to SEQ ID NO: 43; (r) a nucleotide sequence at least 95% identical to SEQ ID NO: 43; (s) a nucleotide sequence at least 99% identical to SEQ ID NO: 43; and (t) the nucleotide sequence of SEQ ID NO: 43.

In some embodiments, the method further comprises contacting at least one polynucleotide aptamer or a therapeutically effective amount of a polynucleotide aptamer with an antidote. In some embodiments, the antidote is an oligonucleotide comprising a sequence complementary to at least a portion of at least one polynucleotide aptamer or a therapeutically effective amount of a polynucleotide aptamer.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

Having thus described the presently disclosed subject matter in general terms, reference will now be made to the accompanying Figures, which are not necessarily drawn to scale, and wherein:

FIG. 1 is a schematic of SELEX process to select for oxyHbS- and deoxyHbS-binding aptamers. The initial RNA library was subjected to 4 rounds of positive selection in which the aptamer pool was incubated with deoxygenated HbS, followed by recovery of bound aptamers and reverse transcription to generate dsDNA. The dsDNA was amplified and transcribed to create an aptamer pool for the following round. Oxygenated HbS was the target in round 5 and both the bound and unbound fractions were recovered in this round. The bound pool underwent 9 more rounds (6-14) of positive selection against oxygenated HbS. The unbound pool underwent 10 subsequent rounds to select for deoxyHbS-targetting aptamers. Because of the difficulty in maintaining high levels of deoxyHbS in the binding steps, rounds of positive selection (6, 8, 10, 12, 14) against a deoxygenated HbS preparation were alternated with rounds of counter selection (7, 9, 11, 13, 15) against an oxygenated HbS preparation, with the unbound aptamers collected and advanced for further selection in the counter selective cycles.

FIG. 2A, FIG. 2B, FIG. 2C, FIG. 2D, FIG. 2E, FIG. 2F, FIG. 2G, FIG. 2H, and FIG. 2I show inhibition of HbS polymerization by aptamers DE3A and OX3B (FIG. 2A, FIG. 2B, and FIG. 2C) and electron micrographs showing fibers present in the polymerization assays (FIG. 2D, FIG. 2E, FIG. 2F, FIG. 2G, FIG. 2H, and FIG. 2I). FIG. 2A depicts typical progression curve for the polymerization assay. Delay time, T_{d}, is described by the x-intercept of the line defining the exponential growth phase. The time for polymerization is represented by (T_{f}−T_{d}) (Adachi & Askura, 1979). Our assays did not proceed to plateau, presumably because eventually the levels of deoxyHbS in our cuvettes began to decrease, resulting in unreliable absorbance readings at the later time points. Therefore the slope of the line defining the exponential growth phase was utilized as a measurement of the rate of polymerization; FIG. 2B is a representative polymerization assay for DE3A. This experiment was repeated 9 times. P values for delay times: DE3A vs. H₂O, P<0.05; DE3A vs. control 1, P<0.05. P values for slopes: DE3A vs. H₂O, P<0.01; DE3A vs. control 1, P<0.01; and FIG. 2C is a representative polymerization assay for OX3B. This experiment was repeated 6 times. P values for delay times: OX3B vs. H₂O, P<0.01; OX3B vs. control 1, P<0.01. P values for slopes: OX3B vs. H₂O, P<0.01; OX3B vs. control 1, P<0.01. Significance between groups was determined by paired Student t test. FIG. 2D, FIG. 2E, FIG. 2F, FIG. 2G, FIG. 2H, and FIG. 2I are electron micrographs showing fibers present in the polymerization assays. Fibers were fixed and stained for transmission electron microscopy at the 78-minute point of the polymerization assay. The entire surface of each grid was randomly examined and representative micrographs are shown for each sample, at a magnification of (FIG. 2D) 135,000× or (FIG. 2E and FIG. 2F) 180,000× or (FIG. 2G, FIG. 2H, and FIG. 2I) 6,000×. FIG. 2D and FIG. 2G show fibers formed in the polymerization assay with H₂O alone. FIG. 2E and FIG. 2H show fibers formed in the polymerization assay with aptamer DE3A. FIG. 2F and FIG. 2I show fibers formed in the polymerization assay with aptamer OX3B.

FIG. 3A shows saturation binding curves for individual aptamers. FIG. 3A shows binding of DE3A to FmetHbS, K_{d}=1.68 μM; binding of DE3A to oxyHbS, K_{d}=1.74 μM; binding of OX3B to FmetHbS, K_{d}=8.57 μM; binding of OX3B to oxyHbS, K_{d}=3.56 μM. Control aptamers exhibited no binding to either conformation. Each curve represents average of 3 replicates. Dissociation constants determined with Graphpad Prism3 software (Graphpad Software Inc., La Jolla, Calif.) for total saturation binding: Specific=X×BMAX/(K_{d}+X); FIG. 3B shows the secondary structure of DE3A (SEQ ID NO. 9) and FIG. 3C shows the secondary structure of OX3B (SEQ ID NO:43), as predicted by Mfold software (Zuker, 2003).

FIG. 4 shows the effect of DE3A or OX3B binding on the oxyhemoglobin dissociation curve. Sickle hemoglobin lysate was combined with aptamer at an aptamer:heme ratio of 1:10, or with water as a control, and analyzed on a Hemox Analyzer. A representative experiment is shown; curves for DE3A, OX3B and water control overlap. Means for p50 values: DE3A=15.49, OX3B=15.53, H₂O=15.52. P values for p50 values: DE3A vs. H₂O, P=0.8713; OX3B vs. H₂O, P=0.9712. Analysis was run in triplicate and significance between groups was determined by paired Student t test.

FIG. 5A, FIG. 5B, FIG. 5C, and FIG. 5D show the response of delay time and polymerization rate of HbS to concentration of aptamer or HbF. HbS was combined with specified concentrations of aptamer or HbF and incubated at 37° C. in the presence of dithionite under hypoxic conditions. Polymerization was measured spectrophotometrically at 700 nm. The polymerization rates, represented by the slopes in exponential growth phase, and delay times were determined from the resulting curves for each concentration of aptamer as described in FIG. 2. FIG. 5A shows the effect of aptamer concentration on slope (presented as the % reduction of maximal slope, where maximal slope is the slope of the line defining the exponential growth phase with no aptamer, and % reduction of maximal slope at each aptamer concentration is calculated as (slope of line with no aptamer—slope of line at given aptamer concentration)/(slope of line with no aptamer)); FIG. 5B shows the effect of aptamer concentration on delay time; FIG. 5C shows the effect of HbF concentration on % reduction of maximal slope, measured as described in FIG. 5A; and FIG. 5D shows the effect of HbF concentration on delay time. Each experiment was run in triplicate. In FIG. 5B and FIG. 5D, the lines represent linear regressions, with the delay times at zero concentration of aptamer or HbF constrained to the actual means.

FIG. 6 shows reduction in the proportion of sickled erythrocytes by internalized DE3A or OX3B. Aptamer was transfected into erythrocytes with Lipofectamine 3000 such that the molar ratio of aptamer to total hemoglobin in the lipofection was 1:10 (aptamer:heme). Transfections proceeded for 22 h at 37° C., followed by exposure to hypoxic conditions for 1 h at 37° C. Cells were subsequently fixed with glutaraldehyde and applied to a microscope slide to determine the proportion of sickled cells. This experiment was run in triplicate and Stata V13.1 statistical software was used to perform an ANOVA with repeated measures. Means shown with 95% confidence intervals.

### DETAILED DESCRIPTION OF THE INVENTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements.

Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The presently disclosed subject matter provides methods for inhibiting sickling of erythrocytes using polynucleotide aptamers that specifically bind to sickle hemoglobin (HbS) in the erythrocytes. The polynucleotide aptamers are capable of inhibiting sickling in HbS-containing erythrocytes, for example, by extending the delay time and reducing the rate of polymerization of HbS, and may be useful as therapeutic agents for sickle cell disease.

### I. Methods of Inhibiting Sickling of an Erythrocyte

Aspects of the presently disclosed subject matter relate to inhibiting sickling of erythrocytes. Generally, sickling of erythrocytes can be inhibited by inhibiting polymerization of a first sickle hemoglobin (HbS) and a second HbS, for example, by specifically binding at least one presently disclosed polynucleotide aptamer to the first HbS (or the second HbS), thereby inhibiting polymerization of the first HbS and the second HbS and inhibiting sickling of the erythrocyte comprising the first HbS and the second HbS. In some embodiments, binding of at least one presently disclosed polynucleotide aptamer to the first HbS (or the second HbS) occurs prior to deoxygenation of the first HbS (or the second HbS). In some embodiments, binding of at least one presently disclosed polynucleotide aptamer to the first HbS (or the second HbS) occurs during or after deoxygenation of the first HbS (or the second HbS). In some embodiments, it has been found that the presently disclosed aptamers bind to both oxygenated and deoxygenated hemoglobin with similar affinity.

In some embodiments, it has been found that the time required for stable nuclei to form following deoxygenation of the HbS (i.e., delay time) increases when at least one polynucleotide aptamer is bound to the HbS. Additionally, in some embodiments, once HbS polymerization is initiated, at least one polynucleotide aptamer slows the rate of HbS polymer formation and, in some cases, the extent of HbS polymer formation.

Accordingly, in some embodiments, the presently disclosed subject matter provides a method for inhibiting sickling of an erythrocyte, the method comprising introducing at least one polynucleotide aptamer into an erythrocyte comprising at least a first sickle hemoglobin (HbS) and a second HbS under conditions effective to specifically bind the at least one polynucleotide aptamer to the first HbS, wherein specifically binding the at least one polynucleotide aptamer to the first HbS inhibits polymerization of the first HbS with the second HbS, thereby inhibiting sickling of the erythrocyte.

Normally, hemoglobin is a tetrameric protein composed of two pairs of two different subunits. Hemoglobin A (hereinafter abbreviated as HbA) has α-chain and β-chain subunits. Binding of glucose to N-terminal amino acid(s) of this/these β-chain results in hemoglobin A_{1c} (hereinafter abbreviated as HbA_{1c}). HbA_{1c}, if produced via a reversible reaction there between, is called labile HbA_{1c} and, if produced via an irreversible reaction involving the labile HbA_{1c}, is called stable HbA_{1c}.

The separation of hemoglobins present in a hemolyzed sample by means of cation exchange liquid chromatography, if performed over a sufficiently long period of time, generally results in the sequential elution of hemoglobin Ala (hereinafter abbreviated as HbA₁ₐ) and hemoglobin A_{1b} (hereinafter abbreviated as HbA_{1b}), hemoglobin F (hereinafter abbreviated as HbF), labile HbA_{1c}, stable HbA_{1c} and hemoglobin A₀ (hereinafter abbreviated as HbA₀). HbA₁ₐ, HbA_{1b} and HbA_{1c} each is a glycated HbA. HbF is fetal hemoglobin composed of α and γ chains. HbA₀ consists of a group of hemoglobin components, includes HbA as its primary component and is retained more strongly to a column than HbA_{1c}.

Hemoglobin S or sickle hemoglobin (hereinafter abbreviated as HbS) and hemoglobin C (hereinafter abbreviated as HbC) are known as “abnormal hemoglobins.” HbS and HbC result from substitution of glutamic acid located in a sixth position from an N-terminal of the β chain of HbA for valine and lysine, respectively. Hemoglobin A₂ (hereinafter abbreviated as HbA₂) is composed of α and δ chains and, like HbF, its elevated level is interpreted as evidence of Mediterranean anemia (thalassemia). In the normal determination of hemoglobins by cation exchange liquid chromatography, they are eluted in the sequence of HbA₀, HbA₂, HbS and HbC.

As used herein, the term “sickling” refers to the process whereby a normal-shaped cell becomes crescent-shaped. As used herein, a “sickle cell” includes a cell which is an abnormal, crescent-shaped erythrocyte that contains sickle cell hemoglobin (HbS). “Erythrocytes”, also called red blood cells, are the most common type of blood cell and are rich in hemoglobin, an iron-containing biomolecule that can bind oxygen.

It is understood by those of skill in the art that a 100% inhibiting of sickling is not required within the presently disclosed methods. In some embodiments, the presently disclosed methods inhibit the sickling of erythrocytes at least about 10%, 15%, 20%, 25%, 30%, 35%, 40%, 45%, 50%, 55%, 60%, 65%, 70%, 75%, 80%, 85%, 90%, 95%, or even 100% relative to sickling of erythrocytes measured in absence of aptamers or with modified aptamers, i.e., a control sample, in an assay. In some embodiments, sickling of erythrocytes is inhibited at least about 20% compared to the sickling in erythrocytes in the absence of aptamers or with modified aptamers, i.e., a control sample. In some embodiments, sickling of erythrocytes is inhibited at least about 30% compared to the sickling in erythrocytes in the absence of aptamers or with modified aptamers, i.e., a control sample.

Aptamers are small single-stranded nucleic acid molecules (˜5-25 kDa) that fold into unique structures, allowing them to bind to molecular targets with high specificity and affinity. This specific binding confers the potential for aptamers to be used in a wide variety of diagnostic or therapeutic applications and have emerged as viable alternatives to small-molecule and antibody-based therapy (Que-Gewirth & Sullenger, 2007; Sun & Zu, 2015; Sundaram et al, 2013). Like antibodies, aptamers possess binding affinities in the low nanomolar to picomolar range. However, aptamers are advantageous in that they are easily synthesized and stored, can bind very small targets, are non-immunogenic, are heat stable, possess minimal interbatch variability, and can be antidote-controlled. Furthermore, chemical modifications, such as amino or fluoro substitutions at the 2′ position of pyrimidines, may reduce degradation by nucleases. The biodistribution and clearance of aptamers can also be altered by chemical addition of moieties such as polyethylene glycol and cholesterol.

An aptamer's small size also maximizes its ability to bind to a specific site on a protein, altering the function of that site, without affecting the functions of other sites on the protein. For example, Fortenberry and colleagues have developed aptamers that bind specifically to plasminogen activator inhibitor-1 (PAI-1), a serine protease inhibitor that has a role in the pathophysiology of several diseases, including cancer and cardiovascular disease. PAI-1 binds to vitronectin, preventing vitronectin's interaction with integrin, thereby resulting in a decrease in cell adhesion and migration. These aptamers bind specifically to PAI-1's vitronectin binding site, affecting PAI-1's interaction with vitronectin, but having no effect on its proteolytic activity.

Specific aptamers are typically selected from very large libraries of more than 10¹⁴ random sequence oligonucleotides in a process called the “systematic evolution of ligands by exponential enrichment” (SELEX). This is an iterative selection process, which begins with a protein or other target of interest being incubated with the oligonucleotide library. A small fraction of the oligonucleotides bind the target and the rest are separated out by a suitable separation technique. The small population that bound the target is then amplified and used in the next round of incubation with the target. This cycle is repeated multiple times, with increasingly stringent incubation and separation conditions at each round in order to enrich for high affinity binders.

The sequence of the polynucleotide aptamers of the presently disclosed subject matter may be selected by any method known in the art. In some embodiments, aptamers are selected by the SELEX process. In some embodiments, aptamers may be selected by starting with the sequences and structural requirements of the aptamers disclosed herein and modifying the sequences to produce other aptamers.

As used herein, the term “polynucleotide aptamer” refers to an aptamer that comprises a number of nucleotide units. The length of the aptamers of the presently disclosed subject matter is not limited, but typical aptamers have a length of about 10 to about 120 nucleotides, particularly about 80 nucleotides. In certain embodiments, the aptamer may have additional nucleotides attached to the 5′- and/or 3′ end. The additional nucleotides may be, e.g., part of primer sequences, restriction endonuclease sequences, or vector sequences useful for producing the aptamer.

The polynucleotide aptamers of the presently disclosed subject matter may comprise ribonucleotides only (RNA aptamers), deoxyribonucleotides only (DNA aptamers), or a combination of ribonucleotides and deoxyribonucleotides. The nucleotides may be naturally occurring nucleotides (e.g., ATP, TTP, GTP, CTP, UTP) or modified nucleotides. Modified nucleotides refers to nucleotides comprising bases such as, for example, adenine, guanine, cytosine, thymine, and uracil, xanthine, inosine, and queuosine that have been modified by the replacement or addition of one or more atoms or groups. Some examples of types of modifications that can comprise nucleotides that are modified with respect to the base moieties, include but are not limited to, alkylated, halogenated, thiolated, aminated, amidated, or acetylated bases, in various combinations. More specific examples include 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety (e.g., 2′-fluoro or 2′-O-methyl nucleotides), as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. The term nucleotide is also meant to include what are known in the art as universal bases. By way of example, universal bases include but are not limited to 3-nitropyrrole, 5-nitroindole, or nebularine. Modified nucleotides include labeled nucleotides such as radioactively, enzymatically, or chromogenically labeled nucleotides. In some embodiments, at least one polynucleotide aptamer is an RNA aptamer.

As used herein, “polymerization” includes the process of forming a polymer from many monomeric units of hemoglobin. A polymer may be formed by any chemical bonding interaction between or among molecules, i.e. covalent, ionic, or van der Waals. As used herein, “aggregation” and “polymerization” may be used interchangeably. In particular embodiments, the presently disclosed aptamers inhibit polymerization of HbS. However, it is understood by those of skill in the art that 100% inhibition of polymerization of HbS is not required within the presently disclosed methods. In some embodiments, the presently disclosed methods produce at least about a 10%, 15%, 20%, 25%, 30%, 35%, 40%, 45%, 50%, 55%, 60%, 65%, 70%, 75%, 80%, 85%, 90%, 95%, or even 100% inhibition of polymerization of HbS relative to polymerization of HbS measured in the absence of aptamers or with modified aptamers described herein that specifically bind HbS and inhibit polymerization of HbS, i.e., a control sample, in an assay. In some embodiments, the HbS, such as the first HbS and/or the second HbS, is a monomer. In some embodiments, the HbS, such as the first HbS and/or the second HbS, is a polymer.

The term “specifically binds,” as used herein, refers to a molecule (e.g., an aptamer) that binds to a target (e.g., a protein such as HbS) with at least five-fold greater affinity as compared to any non-targets, e.g., at least 10-, 20-, 50-, or 100-fold greater affinity. The aptamers of the presently disclosed subject matter may bind HbS, including oxy-HbS and/or deoxy-HbS, as well as HbA and other types of hemoglobin, with a K_{d} of less than about 10 μM, e.g., less than about 5, 2, 1, 0.5, or 0.2 μM.

The methods described herein for inhibiting sickling of an erythrocyte may be carried out using a single aptamer targeted to HbS, or may be carried out using two or more different aptamers targeted to HbS, e.g., three, four, five, or six different aptamers.

As used herein, the term “inhibit” means to decrease, suppress, attenuate, diminish, or arrest the activity of a biological pathway or a biological activity such as polymerization of HbS, e.g., by at least 10%, 20%, 30%, 40%, 50%, 60%, 70%, 80%, 90%, 95%, 98%, 99%, or even 100% compared to an untreated control biological pathway or biological activity.

In some embodiments, specifically binding at least one polynucleotide aptamer to the first HbS inhibits polymerization of the first HbS with a second HbS without affecting the oxygen affinity of the first HbS. As used herein, the term “oxygen affinity” refers to how readily a molecule, such as hemoglobin, acquires and releases oxygen molecules.

In some embodiments, specifically binding at least one polynucleotide aptamer to the HbS, such as the first HbS, occurs under physiological conditions, such as those that occur in a cell system or an organism. In some embodiments, specifically binding at least one polynucleotide aptamer to the HbS, such as the first HbS, occurs under hypoxic conditions. As used herein, the term “hypoxic conditions” refers to conditions where the oxygen level is lower than normal, such as less than 21%, 15%, 12%, 9%, 6%, 3%, or 2%.

In some embodiments, specifically binding at least one polynucleotide aptamer to the first HbS increases the delay time before polymerization of the first HbS with the second HbS occurs. In some embodiments, specifically binding at least one polynucleotide aptamer to the first HbS increases the delay time before polymerization of the first HbS with the second HbS occurs by at least about 2-fold, 3-fold, 4-fold, 5-fold, 6-fold, 7-fold, 8-fold, 9-fold, or 10-fold.

In some embodiments, specifically binding at least one polynucleotide aptamer to the first HbS reduces the rate of polymerization of the first HbS with the second HbS. In some embodiments, specifically binding at least one polynucleotide aptamer to the first HbS reduces the rate of polymerization of the first HbS with the second HbS by at least about 60%, about 70%, about 80%, or 90%. As used herein, the term “rate of polymerization” refers to the speed at which polymerization happens.

In some embodiments, at least one polynucleotide aptamer specifically binds oxygenated HbS. In some embodiments, at least one polynucleotide aptamer specifically binds deoxygenated HbS. In some embodiments, at least one polynucleotide aptamer specifically binds both oxygenated HbS and deoxygenated HbS. In some embodiments, at least one polynucleotide aptamer specifically binds both oxygenated HbS and deoxygenated HbS with similar affinity.

In some embodiments, at least one polynucleotide aptamer consists of from about 70 to about 90 nucleotides. In some embodiments, at least one polynucleotide aptamer consists of about 80 nucleotides. In some embodiments, at least one polynucleotide aptamer comprises the nucleotide sequence of 5′-GGGAGGACGAUGCGG(N40)CAGACGACUCGCUGAGGAUCCGAGA-3′, where (N40) is a variable region. In some embodiments, at least one polynucleotide aptamer forms a stem-loop (hairpin) structure. In some embodiments, at least one polynucleotide aptamer forms at least one stem-loop structure, such as 1 stem-loop structure, 2 stem-loop structures, 3 stem-loop structures, 4 stem-loop structures, or 5 stem-loop structures.

As used herein, the term “stem-loop structure” refers to the secondary structure formed in a nucleic acid when two regions of the same strand, usually complementary when read in opposite directions, base-pair to form a double helix that ends in an unpaired loop.

In some embodiments, at least one polynucleotide aptamer comprises a nucleotide sequence selected from the group consisting of: (a) a nucleotide sequence at least 80% identical to any one of SEQ ID NOs: 7-66; (b) a nucleotide sequence at least 90% identical to any one of SEQ ID NOs: 7-66; (c) a nucleotide sequence at least 95% identical to any one of SEQ ID NOs: 7-66; (d) a nucleotide sequence at least 99% identical to any one of SEQ ID NOs: 7-66; and (e) the nucleotide sequence of any one of SEQ ID NOs: 7-66. In some embodiments, at least one polynucleotide aptamer comprises a nucleotide sequence selected from the group consisting of: (a) a nucleotide sequence at least 80% identical to the variable region of any one of SEQ ID NOs: 7-66; (b) a nucleotide sequence at least 90% identical to the variable region of any one of SEQ ID NOs: 7-66; (c) a nucleotide sequence at least 95% identical to the variable region of any one of SEQ ID NOs: 7-66; (d) a nucleotide sequence at least 99% identical to the variable region of any one of SEQ ID NOs: 7-66; and (e) the nucleotide sequence of the variable region of any one of SEQ ID NOs: 7-66.

In some embodiments, specifically binding at least one polynucleotide aptamer to the first HbS reduces the rate and extent of polymerization of the first HbS with the second HbS. In some embodiments, specifically binding at least one polynucleotide aptamer to the first HbS reduces the rate and extent of polymerization of the first HbS with the second HbS, wherein at least one polynucleotide aptamer comprises a nucleotide sequence selected from the group consisting of: (a) a nucleotide sequence at least 80% identical to SEQ ID NO: 1; (b) a nucleotide sequence at least 90% identical to SEQ ID NO: 1; (c) a nucleotide sequence at least 95% identical to SEQ ID NO: 1; (d) a nucleotide sequence at least 99% identical to SEQ ID NO: 1; (e) the nucleotide sequence of SEQ ID NO: 1; (f) a nucleotide sequence at least 80% identical to SEQ ID NO: 9; (g) a nucleotide sequence at least 90% identical to SEQ ID NO: 9; (h) a nucleotide sequence at least 95% identical to SEQ ID NO: 9; (i) a nucleotide sequence at least 99% identical to SEQ ID NO: 9; and (j) the nucleotide sequence of SEQ ID NO: 9. As used herein, the term “extent of polymerization” refers to the amount or degree to which polymerization occurs.

In some embodiments, specifically binding at least one polynucleotide aptamer to the first HbS reduces the rate of polymerization without reducing the extent of polymerization of the first HbS with the second HbS. In some embodiments, specifically binding at least one polynucleotide aptamer to the first HbS reduces the rate of polymerization without reducing the extent of polymerization of the first HbS with the second HbS, wherein at least one polynucleotide aptamer comprises a nucleotide sequence selected from the group consisting of: (a) a nucleotide sequence at least 80% identical to SEQ ID NO: 2; (b) a nucleotide sequence at least 90% identical to SEQ ID NO: 2; (c) a nucleotide sequence at least 95% identical to SEQ ID NO: 2; (d) a nucleotide sequence at least 99% identical to SEQ ID NO: 2; and (e) the nucleotide sequence of SEQ ID NO: 2; (f) a nucleotide sequence at least 80% identical to SEQ ID NO: 43; (g) a nucleotide sequence at least 90% identical to SEQ ID NO: 43; (h) a nucleotide sequence at least 95% identical to SEQ ID NO: 43; (i) a nucleotide sequence at least 99% identical to SEQ ID NO: 43; and the nucleotide sequence of SEQ ID NO: 43.

In some embodiments, at least one polynucleotide aptamer comprises a nucleotide sequence that is at least 70% identical, e.g., at least 75%, 80%, 85%, 90%, 91%, 92%, 93%, 94%, 95%, 96%, 97%, 98%, or 99% identical to any one of SEQ ID NOs: 1, 2, and 7-66 and the variable regions of SEQ ID NOs: 7-66. In some embodiments, the aptamer consists of a nucleotide sequence that is at least 70% identical, e.g., at least 75%, 80%, 85%, 90%, 91%, 92%, 93%, 94%, 95%, 96%, 97%, 98%, or 99% identical to any one of SEQ ID NOs: 1, 2, and 7-66 and the variable regions of SEQ ID NOs: 7-66. In some embodiments, the aptamer comprises a nucleotide sequence that is identical to a fragment of any one of SEQ ID NOs: 1, 2, and 7-66 and the variable regions of SEQ ID NOs: 7-66 of at least 10 contiguous nucleotides, e.g., at least about 15, 20, 25, 30, or 35 contiguous nucleotides. In some embodiments, the aptamer comprises a nucleotide sequence that is at least 70% identical, e.g., at least 75%, 80%, 85%, 90%, 91%, 92%, 93%, 94%, 95%; 96%, 97%, 98%, or 99% identical to a fragment of any one of SEQ ID NOs: 1, 2, and 7-66 and the variable regions of SEQ ID NOs: 7-66 of at least contiguous 10 nucleotides, e.g., at least about 15, 20, 25, 30, or 35 contiguous nucleotides. In some embodiments, the polynucleotide aptamers described herein comprise both ribonucleotides and deoxyribonucleotides. In some embodiments, the fragments and/or analogs of the aptamers of SEQ ID NOs: 1, 2, and 7-66 and the variable regions of SEQ ID NOs: 7-66 have a substantially similar activity as one or more of the aptamers of SEQ ID NOs: 1, 2, and 7-66 and the variable regions of SEQ ID NOs: 7-66.

“Substantially similar,” as used herein, refers to specific binding to HbS, and in some embodiments also refers to an inhibitory activity on the polymerization of HbS, particularly without a deleterious effect on hemoglobin's functional capabilities, that is at least about 20% of the inhibitory activity of one or more of the aptamers of SEQ ID NOs: 1, 2, and 7-66 and the variable regions of SEQ ID NOs: 7-66.

Changes to the aptamer sequences, such as SEQ ID NOs: 1, 2, and 7-66 and the variable regions of SEQ ID NOs: 7-66, may be made based on structural requirements for binding of the aptamers to HbS, including oxy-HbS and/or deoxy-HbS. The structural requirements may be readily determined by one of skill in the art by analyzing common sequences between the disclosed aptamers and/or by mutating the disclosed aptamers and measuring HbS binding affinity.

When a number of individual, distinct aptamer sequences for a single target molecule have been obtained and sequenced as described herein, the sequences can be examined for “consensus sequences.” As used herein, “consensus sequence” refers to a nucleotide sequence or region (which might or might not be made up of contiguous nucleotides) that is found in one or more regions of at least two aptamers, the presence of which can be correlated with aptamer-to-target-binding or with aptamer structure.

A consensus sequence can be as short as three nucleotides long. It also can be made up of one or more noncontiguous sequences with nucleotide sequences or polymers of hundreds of bases long interspersed between the consensus sequences. Consensus sequences can be identified by sequence comparisons between individual aptamer species, which comparisons can be aided by computer programs and other tools for modeling secondary and tertiary structure from sequence information. Generally, the consensus sequence will contain at least about 5 to 20 nucleotides, more commonly from 11 to 15 nucleotides.

As used herein, a “nucleic acid” or “polynucleotide” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

The term “fragment” refers to a nucleotide sequence of reduced length relative to the reference nucleic acid and comprising, over the common portion, a nucleotide sequence identical to the reference nucleic acid. Such a nucleic acid fragment according to the presently disclosed subject matter may be, where appropriate, included in a larger polynucleotide of which it is a constituent. Such fragments comprise, or alternatively consist of, oligonucleotides ranging in length from at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000 or 1500 consecutive nucleotides of a nucleic acid according to the presently disclosed subject matter.

The term “percent identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences may be performed using the Clustal method of alignment (Higgins and Sharp (1989) CABIOS. 5:151-153) with the default parameters, including default parameters for pairwise alignments.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al. (1990) J. Mol. Biol. 215:403-410, and DNASTAR (DNASTAR, Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

Once an aptamer sequence, according to the presently disclosed subject, matter is identified, the aptamer may by synthesized by any method known to those of skill in the art. In some embodiments, aptamers may be produced by chemical synthesis of oligonucleotides and/or ligation of shorter oligonucleotides. In some embodiments, polynucleotides may be used to express the aptamers, e.g., by in vitro transcription, polymerase chain reaction amplification, or cellular expression. The polynucleotides may be DNA and/or RNA and may be single-stranded or double-stranded. In some embodiments, the polynucleotide is a vector which may be used to express the aptamer. The vector may be, e.g., a plasmid vector or a viral vector and may be suited for use in any type of cell, such as mammalian, insect, plant, fungal, or bacterial cells. The vector may comprise one or more regulatory elements necessary for expressing the aptamers, e.g., a promoter, enhancer, transcription control elements, etc.

Several methods known in the art may be used to propagate a polynucleotide according to the presently disclosed subject matter. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes both viral and nonviral means for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids such as pBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include but are not limited to retrovirus, adeno-associated virus, pox, baculovirus, vaccinia, herpes simplex, Epstein-Barr, adenovirus, geminivirus, and caulimovirus vectors. Non-viral vectors include plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

As used herein, the term “introduced”, such as “introduced into an erythrocyte”, refers to the delivery of a molecule, such as a polynucleotide aptamer, into a cell, such as an erythrocyte. Vectors comprising the presently disclosed polynucleotide aptamers may be introduced into the desired host cells, such as erythrocytes, by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al. (1992) J. Biol. Chem. 267:963; Wu et al. (1988) J. Biol. Chem. 263:14621). Aptamers may also be targeted to cells of interest by coupling aptamers to other aptamers that are known to specifically enter cells of interest, which can be screened for, or by attachment to other ligands for red cell receptors that are internalized (e.g., transferrin-transferrin receptors), as described more fully below.

Polynucleotides can also be introduced in vivo by lipofection. For the past decade, there has been increasing use of liposomes for encapsulation and transfection of nucleic acids in vitro. Synthetic cationic lipids designed to limit the difficulties and dangers encountered with liposome-mediated transfection can be used to prepare liposomes for in vivo transfection of a gene encoding a marker (Feigner et al. (1988) Proc. Natl. Acad. Sci. USA 84:7413; Mackey et al. (1988) Proc. Natl. Acad. Sci. U.S.A. 85:8027; and Ulmer et al. (1993) Science 259:1745). The use of cationic lipids may promote encapsulation of negatively charged nucleic acids, and also promote fusion with negatively charged cell membranes (Feigner et al. (1989) Science 337:387). Particularly useful lipid compounds and compositions for transfer of nucleic acids are described in PCT Patent Pubs. WO95/18863 and WO96/17823, and in U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (Mackey et al. (1988) Proc. Natl. Acad. Sci. U.S.A. 85:8027). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., PCT Patent Pub. WO95/21931), peptides derived from DNA binding proteins (e.g., PCT Patent Pub. WO96/25508), or a cationic polymer (e.g., PCT Patent Pub. WO95/21931).

It is also possible to introduce a vector in vivo as a naked DNA plasmid (see U.S. Pat. Nos. 5,693,622, 5,589,466 and 5,580,859). Receptor-mediated DNA delivery approaches can also be used (Curiel et al. (1992) Hum. Gene Ther. 3:147; Wu et al. (1987) J. Biol. Chem. 262:4429).

The term “transfection” means the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

In some embodiments, at least one polynucleotide aptamer is modified to prevent nuclease degradation. In some embodiments, at least one polynucleotide aptamer is modified to increase the circulating half-life of the aptamer after administration to a subject. In some embodiments, the nucleotides of the aptamers are linked by phosphate linkages. In some embodiments, one or more of the intemucleotide linkages are modified linkages, e.g., linkages that are resistant to nuclease degradation. The term “modified intemucleotide linkage” includes all modified intemucleotide linkages known in the art or that come to be known and that, from reading this disclosure, one skilled in the art will conclude is useful in connection with the presently disclosed methods. Intemucleotide linkages may have associated counterions, and the term is meant to include such counterions and any coordination complexes that can form at the intemucleotide linkages. Modifications of intemucleotide linkages include, without limitation, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that can be saturated or unsaturated and/or substituted and/or contain heteroatoms, linkages with morpholino structures, amides, polyamides wherein the bases can be attached to the aza nitrogens of the backbone directly or indirectly, and combinations of such modified intemucleotide linkages. In another embodiment, the aptamers comprise 5′- or 3′-terminal blocking groups to prevent nuclease degradation (e.g., an inverted deoxythymidine or hexylamine).

In a further embodiment, the aptamers are linked to conjugates that increase the circulating half-life, e.g., by decreasing nuclease degradation or renal filtration of the aptamer. Conjugates may include, for example, amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of conjugates also include steroids, such as cholesterol, phospholipids, di- and tri-acylglycerols, fatty acids, hydrocarbons that may or may not contain unsaturation or substitutions, enzyme substrates, biotin, digoxigenin, and polysaccharides. Further examples include thioethers such as hexyl-S-tritylthiol, thiocholesterol, acyl chains such as dodecandiol or undecyl groups, phospholipids such as di-hexadecyl-rac-glycerol, triethylammonium 1,2-di-O-hexadecyl-rac-glycero-3-H-phosphonate, polyamines, polyethylene glycol, adamantane acetic acid, palmityl moieties, octadecylamine moieties, hexylaminocarbonyl-oxycholesterol, famesyl, geranyl and geranylgeranyl moieties, such as polyethylene glycol, cholesterol, lipids, or fatty acids. Conjugates can also be detectable labels. For example, conjugates can be fluorophores. Conjugates can include fluorophores such as TAMRA, BODIPY, cyanine derivatives such as Cy3 or Cy5 Dabsyl, or any other suitable fluorophore known in the art. A conjugate may be attached to any position on the terminal nucleotide that is convenient and that does not substantially interfere with the desired activity of the aptamer that bears it, for example the 3′ or 5′ position of a ribosyl sugar. A conjugate substantially interferes with the desired activity of an aptamer if it adversely affects its functionality such that the ability of the aptamer to bind HbS, including oxy-HbS and/or deoxy-HbS, is reduced by greater than 80% in a binding assay.

In a further embodiment, the aptamers as described herein that specifically bind HbS are linked to conjugates to mediate intracellular delivery into a cell of interest. “Cell of interest” as used herein refers to red blood cells (RBCs or erythrocytes) and include nucleated or non-nucleated adult and/or fetal red blood cells, but may also refer to erythroblasts, reticulocytes, and/or normoblasts. Such conjugates that mediate intracellular delivery of the aptamers as described herein that specifically bind HbS into a cell of interest include other aptamers that are known to specifically enter cells of interest (referred to herein as “delivery aptamers”) or other ligands that bind receptors on a cell of interest and are internalized by the cell (e.g., transferrin and transferrin receptors (CD71) on red blood cells). Such conjugates may further include detectable labels such as fluorophores to facilitate methods of screening cells of interest containing the aptamers as described herein that specifically bind HbS.

Where the conjugates are delivery aptamers, the delivery aptamers and the aptamers as described herein that specifically bind HbS may be linked, for example, covalently or functionally through nucleic acid duplex formation. At least one of the linked aptamers may be partly or wholly comprised of 2′-modified RNA or DNA such as 2′F, 2′OH, 2′OMe, 2′allyl, 2′MOE (methoxy-O-methyl) substituted nucleotides, and may contain polyethylene glycol (PEG)-spacers and abasic residues. Covalent linkages for delivery aptamers and other ligands may include, for example, a linking moiety such as a nucleic acid moiety, a PNA moiety, a peptidic moiety, a disulfide bond or a polyethylene glycol (PEG) moiety. In some embodiments, at least one polynucleotide aptamer comprises at least one 2′-fluoro nucleotide.

### II. Methods of Treating or Preventing Sickle Cell Disease

In some embodiments, the presently disclosed subject matter provides methods for treating or preventing sickle cell disease in a subject using the presently disclosed polynucleotide aptamers to inhibit or prevent sickling of erythrocytes in the subject by inhibiting HbS polymerization.

Sickle-cell disease (SCD), or sickle-cell anemia (or drepanocytosis), is a life-long blood disorder characterized by red blood cells (erythrocytes: RBC) that assume an abnormal, rigid, sickle shape. Sickling decreases flexibility of RBC and results in a risk of various complications. RBC sickling occurs because of a mutation in the hemoglobin gene. SCD is an inherited disorder and SCD is an autosomal recessive disease. Although, some people who inherit one sickle cell gene and one other defective hemoglobin gene may experience a similar sickle-cell disorder. As used herein, the term “sickle cell disease” includes but is not limited to sickle cell anemia, sickle β-thalassemia, sickle cell-hemoglobin C disease and any other sickle hemoglobinopathy in which HbS interacts with a hemoglobin other than HbS. “Sickle hemoglobinopathy” is an abnormality of hemoglobin which results in sickle cell disease or sickle variants.

In some embodiments, the presently disclosed subject matter provides a method for treating or preventing sickle cell disease in a subject in need thereof, the method comprising administering to a subject a therapeutically effective amount of a polynucleotide aptamer, wherein the polynucleotide aptamer specifically binds to a first HbS in an erythrocyte in the subject, wherein specifically binding the polynucleotide aptamer to the first HbS inhibits polymerization of the first HbS with a second HbS in the erythrocyte, thereby inhibiting sickling of the erythrocyte and treating or preventing sickle cell disease in the subject. In some embodiments, the polynucleotide aptamer is delivered into the erythrocyte.

In some embodiments, the polynucleotide aptamer comprises a nucleotide sequence selected from the group consisting of: (a) a nucleotide sequence at least 80% identical to SEQ ID NO: 1; (b) a nucleotide sequence at least 90% identical to SEQ ID NO: 1; (c) a nucleotide sequence at least 95% identical to SEQ ID NO: 1; (d) a nucleotide sequence at least 99% identical to SEQ ID NO: 1; (e) the nucleotide sequence of SEQ ID NO: 1; (f) a nucleotide sequence at least 80% identical to SEQ ID NO: 9; (g) a nucleotide sequence at least 90% identical to SEQ ID NO: 9; (h) a nucleotide sequence at least 95% identical to SEQ ID NO: 9; (i) a nucleotide sequence at least 99% identical to SEQ ID NO: 9; (j) the nucleotide sequence of SEQ ID NO: 9; (k) a nucleotide sequence at least 80% identical to SEQ ID NO:2; (1) a nucleotide sequence at least 90% identical to SEQ ID NO: 2; (m) a nucleotide sequence at least 95% identical to SEQ ID NO: 2; (n) a nucleotide sequence at least 99% identical to SEQ ID NO: 2; (o) the nucleotide sequence of SEQ ID NO: 2; (p) a nucleotide sequence at least 80% identical to SEQ ID NO: 43; (q) a nucleotide sequence at least 90% identical to SEQ ID NO: 43; (r) a nucleotide sequence at least 95% identical to SEQ ID NO:43; (s) a nucleotide sequence at least 99% identical to SEQ ID NO: 43; and (t) the nucleotide sequence of SEQ ID NO: 43.

In some embodiments, the polynucleotide aptamer is a mixture of more than one kind of polynucleotide aptamer, such as a combination of at least two polynucleotide aptamers comprising two of the nucleotide sequences disclosed herein.

In some embodiments, the polynucleotide aptamer is delivered into an erythrocyte comprising at least a first sickle hemoglobin (HbS) and a second HbS in the subject. In some embodiments, the polynucleotide aptamer is introduced into erythrocytes in vitro or ex vivo. In some embodiments, the polynucleotide aptamer is introduced into erythrocytes and then these erythrocytes are administered to a subject, such as by a red blood cell transfusion, for example. In some embodiments, the polynucleotide aptamer is administered directly to a subject and is introduced into erythrocytes in vivo.

In some embodiments, the presently disclosed subject matter also relates to antidotes for the aptamers that specifically bind to HbS and inhibit polymerization of HbS as disclosed herein. Such antidotes can comprise oligonucleotides that are reverse complements of segments of the aptamers that specifically bind to HbS and inhibit polymerization of HbS as disclosed herein. In accordance with the presently disclosed subject matter, the antidote is contacted with a targeted aptamer under conditions such that it binds to the aptamer and modifies the interaction between the aptamer and its target molecule (e.g., HbS). Modification of that interaction can result from modification of the aptamer structure as a result of binding by the antidote. The antidote can bind free aptamer and/or aptamer bound to its target molecule.

Antidotes of the presently disclosed subject matter can be designed so as to bind any particular aptamer with a high degree of specificity and a desired degree of affinity. The antidote can be designed so that upon binding to the targeted aptamer, the three-dimensional structure of that aptamer is altered such that the aptamer can no longer bind to its target molecule or binds to its target molecule with less affinity.

Antidotes of the presently disclosed subject matter include any pharmaceutically acceptable agent that can bind an aptamer and modify the interaction between that aptamer and its target molecule (e.g., by modifying the structure of the aptamer) in a desired manner. Examples of such antidotes include oligonucleotides complementary to at least a portion of the aptamers that specifically bind to HbS and inhibit polymerization of HbS as disclosed herein (including ribozymes or DNAzymes or peptide nucleic acids), nucleic acid binding peptides, polypeptides or proteins including nucleic acid binding tripeptides (see generally, Hwang et al. (1999) Proc. Natl. Acad. Sci. USA 96:12997), and oligosaccharides such as aminoglycosides (see, generally, Davies et al. (1993) Chapter 8, p. 185, RNA World, Cold Spring Harbor Laboratory Press, eds. Gestlaad and Atkins; Werstuck et al. (1998) Science 282:296; U.S. Pat. Nos. 5,935,776 and 5,534,408; Chase et al. (1986) Ann. Rev. Biochem. 56:103; Eichhorn et al. (1968) J. Am. Chem. Soc. 90:7323; Dale et al. (1975) Biochemistry 14:2447; and Lippard et al. (1978) Acc. Chem. Res. 11:211).

Standard binding assays can be used to screen for antidotes of the presently disclosed subject matter (e.g., using BIACORE assays). Candidate antidotes can be contacted with the aptamer to be targeted under conditions favoring binding and a determination made as to whether the candidate antidote in fact binds the aptamer. Candidate antidotes that are found to bind the aptamer can then be analyzed in an appropriate bioassay (which will vary depending on the aptamer and its target molecule) to determine if the candidate antidote can affect the binding of the aptamer to its target molecule.

Where the antidote is an oligonucleotide, the antidote oligonucleotide does not need to be completely complementary to the aptamer that specifically binds to HbS and inhibits polymerization of HbS as disclosed herein as long as the antidote sufficiently binds to or hybridizes to the aptamer to neutralize its activity. In one embodiment, the antidote of the presently disclosed subject matter is an oligonucleotide that comprises a sequence complementary to at least a portion of the targeted aptamer sequence. In one embodiment, the antidote oligonucleotide comprises a sequence complementary to up to 80, 75, 70, 65, 60, 55, 50, 45, 40, 35, 30, 25, 20, 15, 10, or 5 consecutive nucleotides of the targeted aptamer. In some embodiments, the antidote oligonucleotide comprises a sequence complementary to all but 1, 2, 3, 4, 5, 6, 7, 8, 9, or 10 nucleotides of the targeted aptamer.

In some embodiments, the method further comprises contacting at least one polynucleotide aptamer or a therapeutically effective amount of a polynucleotide aptamer with an antidote. In some embodiments, the antidote is an oligonucleotide comprising a sequence complementary to at least a portion of at least one polynucleotide aptamer or a therapeutically effective amount of a polynucleotide aptamer.

For use within the methods for treating or preventing sickle cell disease in a subject in need thereof, the aptamers described herein that specifically bind to HbS and inhibit polymerization of HbS may optionally be administered in conjunction with other compounds (e.g., therapeutic agents) or treatments (e.g., hydroxyurea or blood transfusions) useful in treating sickle cell disease. The other compounds or treatments may optionally be administered concurrently. As used herein, the word “concurrently” means sufficiently close in time to produce a combined effect (that is, concurrently may be simultaneously, or it may be two or more events occurring within a short time period before or after each other). The other compounds may be administered separately from the aptamers as disclosed herein, or may be combined together with the aptamers as disclosed herein in a single composition.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. The terms “treat,” “treating,” “treatment,” and the like, as used herein can refer to curative therapy, prophylactic therapy, and preventative therapy. Accordingly, as used herein, “treating” means either slowing, stopping or reversing the progression of the sickling of a cell, including reversing the progression to the point of eliminating the presence of sickled cells and/or reducing or eliminating the amount of polymerization of hemoglobin, or the amelioration of symptoms associated with sickle cell disease. Sickle cell disease associated symptoms include, but are not limited to, erythrocyte(RBC) sickling, oxygen release, increased HbS polymerization, hemolysis, tissue congestion and organ damage or dysfunction. The treatment, administration, or therapy can be consecutive or intermittent. Consecutive treatment, administration, or therapy refers to treatment on at least a daily basis without interruption in treatment by one or more days.

Intermittent treatment or administration, or treatment or administration in an intermittent fashion, refers to treatment that is not consecutive, but rather cyclic in nature. Treatment according to the presently disclosed methods can result in complete relief or cure from a disease, disorder, or condition, or partial amelioration of one or more symptoms of the disease, disease, or condition, and can be temporary or permanent. The term “treatment” also is intended to encompass prophylaxis, therapy and cure.

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition. Thus, in some embodiments, an agent can be administered prophylactically to prevent the onset of a disease, disorder, or condition, or to prevent the recurrence of a disease, disorder, or condition.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

In some embodiments, the method of treating sickle cell disease includes a step of selecting a subject for treatment of sickle cell disease. In some embodiments, erythrocytes are selected for treatment using at least one presently disclosed aptamer. In some embodiments, a subject is selected if the subject has sickle cell disease (SCD). In some embodiments, a subject is selected by using a genetic test for detecting the single amino acid substitution of valine for glutamic acid at the 136 position of sickle hemoglobin. In some embodiments, a subject is selected by performing a blood test to see if the erythrocytes in the blood are sickle-shaped. In some embodiments, a subject is selected by performing a blood test to stain for the presence of HbS. In some embodiments, a subject is selected for treatment by testing the erythrocytes of the subject using the presently disclosed polynucleotide aptamers to determine if the aptamers reduce sickling of the erythrocytes and/or inhibit HbS polymerization in the erythrocytes.

The presently disclosed methods may include administering pharmaceutical compositions of aptamers that specifically bind to HbS and inhibit polymerization of HbS as disclosed herein, alone or in combination with one or more additional therapeutic agents, in admixture with a physiologically compatible carrier, which can be administered to a subject, for example, a human subject, for therapeutic or prophylactic treatment. As used herein, “physiologically compatible carrier” refers to a physiologically acceptable diluent including, but not limited to water, phosphate buffered saline, or saline, and, in some embodiments, can include an adjuvant. Acceptable carriers, excipients, or stabilizers are nontoxic to recipients at the dosages and concentrations employed, and can include buffers such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers, such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or nonionic surfactants such as Tween, Pluronics, or PEG. Adjuvants suitable for use with the presently disclosed compositions include adjuvants known in the art including, but not limited to, incomplete Freund's adjuvant, aluminum phosphate, aluminum hydroxide, and alum.

Compositions to be used for in vivo administration must be sterile, which can be achieved by filtration through sterile filtration membranes, prior to or following lyophilization and reconstitution. Therapeutic compositions may be placed into a container having a sterile access port, for example, an intravenous solution bag or vial having a stopper pierceable by a hypodermic injection needle.

In certain embodiments, the presently disclosed subject matter also includes combination therapies. Additional therapeutic agents, which are normally administered to treat or prevent sickle cell disease, may be administered in combination with aptamers that specifically bind to HbS and inhibit polymerization of HbS as disclosed herein. These additional agents may be administered separately, as part of a multiple dosage regimen, from the composition comprising aptamers that specifically bind to HbS and inhibit polymerization of HbS as disclosed herein. Alternatively, these agents may be part of a single dosage form, mixed together with the aptamers that specifically bind to HbS and inhibit polymerization of HbS as disclosed herein, in a single composition.

By “in combination with” is meant the administration of aptamers that specifically bind to HbS and inhibit polymerization of HbS as disclosed herein, with one or more therapeutic agents either simultaneously, sequentially, or a combination thereof. Therefore, a subject administered a combination of aptamers that specifically bind to HbS and inhibit polymerization of HbS as disclosed herein, can receive an aptamer that specifically binds to HbS and inhibits polymerization of HbS as disclosed herein, and one or more therapeutic agents at the same time (i.e., simultaneously) or at different times (i.e., sequentially, in either order, on the same day or on different days), so long as the effect of the combination of both agents is achieved in the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the aptamer that specifically binds to HbS and inhibits polymerization of HbS as disclosed herein, and one or more therapeutic agents are administered simultaneously, they can be administered to the subject as separate pharmaceutical compositions, each comprising either an aptamer that specifically binds to HbS and inhibits polymerization of HbS as disclosed herein, or one or more therapeutic agents, or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered agent is not diminished by the sequential, simultaneous or separate administration of the subsequent agent(s).

The presently disclosed methods may include administering the aptamers using a variety of methods known in the art depending on the subject and the particular disease, disorder, or condition being treated. The administering can be carried out by, for example, intravenous infusion; injection by intravenous, intraperitoneal, intracerebral, intramuscular, intraocular, intraarterial or intralesional routes; or topical or ocular application.

More particularly, as described herein, the presently disclosed aptamers that specifically bind to HbS and inhibit polymerization of HbS can be administered to a subject for therapy by any suitable route of administration, including orally, nasally, transmucosally, ocularly, rectally, intravaginally, parenterally, including intramuscular, subcutaneous, intramedullary injections, as well as intrathecal, direct intraventricular, intravenous, intra-articular, intra-stemal, intra-synovial, intra-hepatic, intralesional, intracranial, intraperitoneal, intranasal, or intraocular injections, intracistemally, topically, as by powders, ointments or drops (including eyedrops), including buccally and sublingually, transdermally, through an inhalation spray, or other modes of delivery known in the art.

The phrases “systemic administration,” “administered systemically,” “peripheral administration” and “administered peripherally” as used herein mean the administration of an aptamer that specifically binds to HbS and inhibits polymerization of HbS, a compound, drug or other material other than directly into the central nervous system, such that it enters the patient's system and, thus, is subject to metabolism and other like processes, for example, subcutaneous administration.

The phrases “parenteral administration” and “administered parenterally” as used herein mean modes of administration other than enteral and topical administration, usually by injection, and includes, without limitation, intravenous, intramuscular, intarterial, intrathecal, intracapsular, intraorbital, intraocular, intracardiac, intradermal, intraperitoneal, transtracheal, subcutaneous, subcuticular, intraarticular, subcapsular, subarachnoid, intraspinal and intrastemal injection and infusion.

Pharmaceutical compositions used in the presently disclosed methods can be manufactured in a manner known in the art, e.g. by means of conventional mixing, dissolving, granulating, dragee-making, levitating, emulsifying, encapsulating, entrapping or lyophilizing processes.

More particularly, pharmaceutical compositions for oral use can be obtained through combination of an aptamer that specifically binds to HbS and inhibits polymerization of HbS with a solid excipient, optionally grinding a resulting mixture, and processing the mixture of granules, after adding suitable auxiliaries, if desired, to obtain tablets or dragee cores.

Suitable excipients include, but are not limited to, carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl cellulose; and gums including arabic and tragacanth; and proteins, such as gelatin and collagen; and polyvinylpyrrolidone (PVP:povidone). If desired, disintegrating or solubilizing agents, such as cross-linked polyvinyl pyrrolidone, agar, alginic acid, or a salt thereof, such as sodium alginate, also can be added to the compositions.

Dragee cores are provided with suitable coatings, such as concentrated sugar solutions, which also can contain gum arabic, talc, polyvinylpyrrolidone, carbopol gel, polyethylene glycol (PEG), and/or titanium dioxide, lacquer solutions, and suitable organic solvents or solvent mixtures. Dyestuffs or pigments can be added to the tablets or dragee coatings for product identification or to characterize the quantity of an aptamer that specifically binds to HbS and inhibits polymerization of HbS, e.g., dosage, or different combinations of aptamer doses.

Pharmaceutical compositions suitable for oral administration include push-fit capsules made of gelatin, as well as soft, sealed capsules made of gelatin and a coating, e.g., a plasticizer, such as glycerol or sorbitol. The push-fit capsules can contain active ingredients admixed with a filler or binder, such as lactose or starches, lubricants, such as talc or magnesium stearate, and, optionally, stabilizers. In soft capsules, the aptamer that specifically binds to HbS and inhibits polymerization of HbS can be dissolved or suspended in suitable liquids, such as fatty oils, liquid paraffin, or liquid polyethylene glycols (PEGs), with or without stabilizers. Stabilizers can be added as warranted.

In some embodiments, pharmaceutical compositions can be administered by rechargeable or biodegradable devices. For example, a variety of slow-release polymeric devices have been developed and tested in vivo for the controlled delivery of drugs, including proteinacious biopharmaceuticals. Suitable examples of sustained release preparations include semipermeable polymer matrices in the form of shaped articles, e.g., films or microcapsules. Sustained release matrices include polyesters, hydrogels, polylactides (U.S. Pat. No. 3,773,919; EP 58,481), copolymers of L-glutamic acid and gamma ethyl-L-glutamate (Sidman et al., Biopolymers 22:547, 1983), poly (2-hydroxyethyl-methacrylate) (Langer et al., J. Biomed. Mater. Res. 15:167, 1981; Langer, Chem. Tech. 12:98, 1982), ethylene vinyl acetate (Langer et al., Id), or poly-D-(−)-3-hydroxybutyric acid (EP 133,988A). Sustained release compositions also include liposomally entrapped aptamers, which can be prepared by methods known per se (Epstein et al., Proc. Natl. Acad. Sci. U.S.A. 82:3688, 1985; Hwang et al., Proc. Natl. Acad. Sci. U.S.A. 77:4030, 1980; U.S. Pat. Nos. 4,485,045 and 4,544,545; and EP 102,324A). Ordinarily, the liposomes are of the small (about 200-800 Angstroms) unilamelar type in which the lipid content is greater than about 30 mol % cholesterol, the selected proportion being adjusted for the optimal therapy. Such materials can comprise an implant, for example, for sustained release of the presently disclosed aptamers that specifically bind to HbS and inhibit polymerization of HbS, which, in some embodiments, can be implanted at a particular, pre-determined target site.

Pharmaceutical compositions for parenteral administration include aqueous solutions of aptamers that specifically bind to HbS and inhibit polymerization of HbS. For injection, the presently disclosed pharmaceutical compositions can be formulated in aqueous solutions, for example, in some embodiments, in physiologically compatible buffers, such as Hank's solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of the aptamers that specifically bind to HbS and inhibit polymerization of HbS or vehicles include fatty oils, such as sesame oil, or synthetic fatty acid esters, such as ethyl oleate or triglycerides, or liposomes. Optionally, the suspension also can contain suitable stabilizers or agents that increase the solubility of the aptamers that specifically bind to HbS and inhibit polymerization of HbS to allow for the preparation of highly concentrated solutions.

For nasal or transmucosal administration generally, penetrants appropriate to the particular barrier to be permeated are used in the formulation. Such penetrants are generallyknown in the art.

For inhalation delivery, the agents of the disclosure also can be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Additional ingredients can be added to compositions for topical administration, as long as such ingredients are pharmaceutically acceptable and not deleterious to the epithelial cells or their function. Further, such additional ingredients should not adversely affect the epithelial penetration efficiency of the composition, and should not cause deterioration in the stability of the composition. For example, fragrances, opacifiers, antioxidants, gelling agents, stabilizers, surfactants, emollients, coloring agents, preservatives, buffering agents, and the like can be present. The pH of the presently disclosed topical composition can be adjusted to a physiologically acceptable range of from about 6.0 to about 9.0 by adding buffering agents thereto such that the composition is physiologically compatible with a subject's skin.

In other embodiments, the pharmaceutical composition can be a lyophilized powder, optionally including additives, such as 1 mM-50 mM histidine, 0.1%-2% sucrose, 2%-7% mannitol at a pH range of 4.5 to 5.5 that is combined with buffer prior to use.

The presently disclosed subject matter also includes the use of aptamers that specifically bind to HbS and inhibit polymerization of HbS disclosed herein, in the manufacture of a medicament for sickle cell disease.

Regardless of the route of administration selected, the presently disclosed aptamers that specifically bind to HbS and inhibit polymerization of HbS, which may be used in a suitable hydrated form, and/or the pharmaceutical compositions are formulated into pharmaceutically acceptable dosage forms such as described below or by other conventional methods known to those of skill in the art.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition (e.g., a disease, condition, or disorder related to polymerization of HbS such as sickle cell disease), or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

Actual dosage levels of the active ingredients in the presently disclosed pharmaceutical compositions can be varied so as to obtain an amount of the active ingredient that is effective to achieve the desired therapeutic response for a particular subject, composition, route of administration, and disease, disorder, or condition without being toxic to the subject. The selected dosage level will depend on a variety of factors including the activity of the particular aptamer employed, the route of administration, the time of administration, the rate of excretion of the particular aptamer being employed, the duration of the treatment, other drugs, aptamers and/or materials used in combination with the particular aptamer employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the aptamers that specifically bind to HbS and inhibit polymerization of HbS, employed in the pharmaceutical composition at levels lower than that required to achieve the desired therapeutic effect and gradually increase the dosage until the desired effect is achieved. Accordingly, the dosage range for administration will be adjusted by the physician as necessary. It will be appreciated that an amount of an aptamer required for achieving the desired biological response, e.g., inhibition of polymerization of HbS, may be different from the amount of compound effective for another purpose.

In general, a suitable daily dose of aptamers that specifically bind to HbS and inhibit polymerization of HbS, will be that amount of the aptamer that is the lowest dose effective to produce a therapeutic effect. Such an effective dose will generally depend upon the factors described above. Generally, doses of the aptamers that specifically bind to HbS and inhibit polymerization of HbS will range from about 0.0001 to about 1000 mg per kilogram of body weight of the subject per day. In certain embodiments, the dosage is between about 1 μg/kg and about 500 mg/kg, more preferably between about 0.01 mg/kg and about 50 mg/kg. For example, in certain embodiments, a dose can be about 1, 5, 10, 15, 20, or 40 mg/kg/day.

If desired, the effective daily dose of the aptamers that specifically bind to HbS and inhibit polymerization of HbS can be administered as two, three, four, five, six or more sub-doses administered separately at appropriate intervals throughout the day, optionally, in unit dosage forms.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments+50%, in some embodiments+20%, in some embodiments+10%, in some embodiments+5%, in some embodiments+1%, in some embodiments+0.5%, and in some embodiments+0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

Identification of Aptamers that Inhibit the Polymerization of Hemoglobin S in Solution and in Sickle Cell Erythrocytes.

Materials and Methods.

Preparation of hemoglobin: In accordance with the requirements of the Johns Hopkins Medicine Institutional Review Board, heparinized venous blood was obtained from discarded blood samples from untransfused homozygous SCD patients. Human blood for HbF experiments was acquired in accordance with the Declaration of Helsinki, with approval by the Institutional Review Board of the Johns Hopkins Hospital School of Medicine. Erythrocytes were washed 5 times with PBS, hemolyzed in 3.5 volumes of distilled water, and stromata were removed by centrifugation at 20,000 g for 25 minutes. Hemoglobin-rich extract was dialyzed into 0.05M tris(hydroxymethyl)aminomethane (tris)-HCl, pH 8.3, and purified HbS was obtained by separation on a DEAE Sephadex A-50 anion exchange column, developing with a gradient of 0.05M tris-HCl, pH 8.3 to 0.05M tris-HCl pH 7.3. The appropriate peak was collected and shown by HPLC to contain 95% HbS. The collected fractions were dialyzed against 2 mM 4-(2-hydroxyethyl)-1-piperazine-1-ethanesulfonic acid (HEPES), pH 7.4 for the SELEX process, and against 1M potassium phosphate buffer, pH 7.1 for use in the polymerization assays, and stored at −80° C. For HbF, hemoglobin-rich extract was acquired as above from a patient with 99.5% HbF, and dialyzed in H2O. Hemoglobin concentrations were measured by Drabkin's method (Van Kampen & Zijlstra, 1983). Proportions of hemoglobin at different oxidation states were determined by the method of Benesch et al., (1973) corrected for an extinction coefficient of 11.0 (Van Assendelft & Zijlstra, 1975).

Cloning, Sequencing and RNA Preparation: At round 11 and following the final round of selection, cDNA was removed for cloning and sequencing of individual aptamers. Sequencing was carried out at the Johns Hopkins Genetic Resources Core Facility. Large quantities of aptamer for further analysis were generated from clone DNA by transcription using the Durascribe T7 kit (Epicentre, Madison, Wis.). RNA was recovered with the RNA Clean and Concentrator-5 kit (Zymo Research, Irvine, Calif.), eluted in H₂O and further concentrated by vacuum when necessary.

Selection of Aptamers Through Systematic Evolution of Ligands by Exponential Enrichment (SELEX): The initial RNA oligonucleotide library comprised the sequence 5′-GGGAGGACGAUGCGG(N40)CAGACGACUCGCUGAGGAUCCGAGA-3′ where N40 represents a random sequence of 40 nucleotides. The RNA incorporated modified nuclease-resistant nucleotides 2′-fluorine-dCTP and 2′-fluorine-dUTP. From this starting RNA library, four initial rounds of selection were performed, in which partially deoxygenated HbS was the target protein. In order to deoxygenate the HbS prior to incubation, the preparation was thawed, exposed to a vacuum by injection into a vacuum tube with a septum cap, and rocked at room temperature for 1 hour. The hemoglobin was then removed from the tube, and incubated with RNA immediately at 37° C. for 5 minutes at a ratio of 3 moles RNA per mole of protein in round 1, increasing to 5 moles RNA per mole of protein by round 4.

Bound RNA was collected by capturing the protein on a nitrocellulose membrane, eluting, and extracting the RNA. Reverse transcription was performed on the eluted RNA followed by PCR. Transcription was performed and the resulting aptamer pool was used in the subsequent selection round. Preliminary tests showed that the vacuum-deoxygenated lysates contain approximately 18-28% deoxyHBS, which decreased during subsequent incubation. The remainder was a mixture of oxygenated HbS (oxyHbS) and methemoglobinS (metHbS); therefore, these first four rounds enriched for aptamers targeting both the T- and R-states. For round 5, oxygenated hemoglobin was the targeted protein. Freshly thawed HbS, in which measurements of the oxidation states showed 81-90% to be oxyHbS, with variable amounts of deoxyHbS and metHbS, was used in the binding reaction. Incubation was carried out at room temperature for 10 minutes at a ratio of 5 moles RNA per mole of protein. Following binding, the aptamer/HbS complexes were captured on nitrocellulose, and the unbound aptamers were also collected and recovered by butanol extraction.

At this point the two collected pools proceeded in two separate selections. The bound pool was utilized to enrich for aptamers that bind to oxyHbS. For this selection, oxygenated HbS was the target in all subsequent rounds (rounds 6-14), and binding was carried out at room temperature for 10 minutes at a ratio of 5 moles RNA per mole of protein in round 6, increasing to 9 moles RNA per mole of protein by round 14. Only bound aptamers were recovered for further selection during these rounds; unbound aptamers were discarded.

The unbound pool from round 5 was used in the second selection, designed to enrich for aptamers that bind to deoxyHbS. In order to remove those aptamers that bind to the oxyHbS still present following deoxygenation, a counter selection was applied: positive selection against deoxyHbS, as described for rounds 1 to 4, was alternated with counter selection against oxyHbS. Bound aptamers were collected for further selection after binding with deoxyHbS, and unbound aptamers were collected for further selection following binding with oxyHbS. Ten rounds were completed in this manner, with binding performed at room temperature for 10 minutes at a ratio of 5 moles RNA per mole of protein in round 6, increasing to 7 moles RNA per mole of protein by round 15. Rounds 1-7 were performed in low-salt binding buffer (20 mM HEPES pH 7.4, 50 mM NaCl, 2 mM CaCl₂, 0.01% BSA) and low-salt wash buffer (20 mM HEPES pH 7.4, 50 mM NaCl, 2 mM CaCl₂). From round 8 on, selections were performed in high-salt binding buffer (20 mM HEPES pH 7.4, 150 mM NaCl, 2 mM CaCl₂, 0.01% BSA) and high-salt wash buffer (20 mM HEPES pH 7.4, 150 mM NaCl, 2 mM CaCl₂).

Table 1. Sequences of unique aptamers identified by SELEX screening. Entire aptamer sequences are shown, including flanking sequences (capitalized). Aptamer name indicates from which pool it was generated (“DE” indicates an aptamer generated from the deoxyHbS-targeting pool, “OX” indicates an aptamer generated from the oxyHbS-targeting pool.) Consensus sequences of at least 10 nucleotides, identified using a ClustalW multiple sequence alignment (Higgins, 1996), are indicated with text formatting: bold, single underline, double underline, or wavy underline. Mutations resulted in the following aptamers having variable regions that were not 40 nt: DE24 (39 nt); 2DE12 (38 nt); OX10 (39 nt); 2OX3 (41 nt); 2OX10 (41 nt).

TABLE 1
Sequences of unique aptamers identified by SELEX screening. Entire aptamer
sequences are shown, including flanking sequences (capitalized). Aptamer name
indicates from which pool it was generated (“DE” indicates an aptamer generated
from the deoxyHbS-targeting pool, “OX” indicates an aptamer generated from the
oxyHbS-targeting pool.) Consensus sequences of at least 10 nucleotides,
identified using a ClustalW multiple sequence alignment (Higgins, 1996), are
indicated with text formatting: bold, single underline, double underline,
or wavy underline. Mutations resulted in the following aptamers having variable
regions that were not 40 nt: DE24 (39 nt); 2DE12 (38 nt); OX10 (39 nt); 2OX3
(41 nt); 2OX10 (41 nt).

| | | Number of |
|---|---|---|
| Aptamer | | times |
| name | Sequence (5′-3′) | represented |
| DE1 | GGGAGGACGAUGCGGccgauuagaacugggcugcgaucggagauccucuag | 1 |
| | guuuCAGACGACUCGCUGAGGAUCCGAGA (SEQ ID NO: 7) | |
| DE2 | GGGAGGACGAUGCGGgccgagggauucgguguagacucugcacaguccuga | 1 |
| | aaagCAGACGACUCGCUGAGGAUCCGAGA (SEQ ID NO: 8) | |
| DE3A | GGGAGGACGAUGCGGccgauuagaacugggcugaggcguucugcauuucgg | 3 |
| | ugauCAGACGACUCGCUGAGGAUCCGAGA (SEQ ID NO: 9) | |
| DE3B | GGGAGGACGAUGCGGccgauuagaacugggcuguuccgacucugaauccgg | 1 |
| | ugauCAGACGACUCGCUGAGGAUCCGAGA (SEQ ID NO: 10) | |
| DE5 | GGGAGGACGAUGCGGuuggugaagggaggucagcauaucuucccgcgggaa | 1 |
| | gcgaCGGACGACUCGCUGAGGAUCCGAGA (SEQ ID NO: 11) | |
| DE7A | GGGAGGACGAUGCGGauccacggguaagggugagggacgacaucaaggcga | 2 |
| | gauuCAGACGACUCGCUGAGGAUCCGAGA (SEQ ID NO: 12) | |
| DE8 | GGGAGGACGAUGCGGuacgauuagaacuggugccgaacagcgcucguugaa | 17 |
| | gacaCAGACGACUCGCUGAGGAUCCGAGA (SEQ ID NO: 13) | |
| DE9 | GGGAGGACGAUGCGGaggaaguaggguucguccauugggcgaguggccugu | 1 |
| | gunaCAGACGACUCGCUGAGGAUCCGAGA (SEQ ID NO: 14) | |
| DE10 | GGGAGGACGAUGCGGcacgguauaguggaguggguaggcaucgcucgacga | 1 |
| | gugaCAGACGACUCGCUGAGGAUCCGAGA (SEQ ID NO: 15) | |
| DE15 | 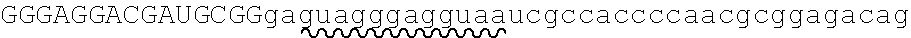 | 1 |
| | cgaCAGACGACUCGCUGAGGAUCCGAGA (SEQ ID NO: 16) | |
| DE19C1 | GGGAGGACGAUGCGGucgauagggggacggaccgcgcuggaaacucaacgua | 1 |
| | gcaCAGACGACUCGCUGAGGAUCCGAGA (SEQ ID NO: 17) | |
| DE20 | GGGAGGACGAUGCGGcacugaugggagugggaucagugucgagcgguaucu | 3 |
| | gcagCAGACGACUCGCUGAGGAUCCGAGA (SEQ ID NO: 18) | |
| DE22 | 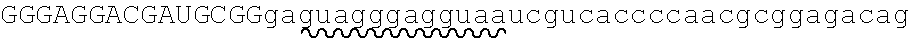 | 2 |
| | cgaCAGACGACUCGCUGAGGAUCCGAGA (SEQ ID NO: 19) | |
| DE24 | GGGAGGACGAUGCGGaagcauacagunuagugugcuagggugggacucagu | 1 |
| | gauCAGACGACUCGCUGAGGAUCCGAGA (SEQ ID NO : 20) | |
| DE28A | GGGAGGACGAUGCGGuccuacuuuccccaauuuguaacagcucuccgcacag | 1 |
| | cagCAGACGACUCGCUGAGGAUCCGAGA (SEQ ID NO: 21) | |
| DE30 | GGGAGGACGAUGCGGcgguguagggaucgucagucucggaaugaccucaca | 1 |
| | gaagCAGACGACUCGCUGAGGAUCCGAGA (SEQ ID NO: 22) | |
| DE31 | GGGAGGACGAUGCGGccagcaggaggaugggugccgcacucggauauucac | 1 |
| | guguCAGACGACUCGCUGAGGAUCCGAGA (SEQ ID NO: 23) | |
| DE33A | GGGAGGACGAUGCGGgacuaagcacaacucaacuagaacgaaccuauuccau | 1 |
| | cauCAGACGACUCGCUGAGGAUCCGAGA (SEQ ID NO: 24) | |
| DE34D | GGGAGGACGAUGCGGaacggaggaguguccucucagcugacagucgugcau | 1 |
| | acuaCAGACGACUCGCUGAGGAUCCGAGA (SEQ ID NO: 25) | |
| DE37A | GGGAGGACGAUGCGGaacucgauccaucaucgugacugcguacgugucaacu | 1 |
| | aagCAGACGACUCGCUGAGGAUCCGAGA (SEQ ID NO: 26) | |
| DE40 | GGGAGGACGAUGCGGgacggucauagagccggccgacauuagagccgggaau | 1 |
| | ccaCAGACGACUCGCUGAGGAUCCGAGA (SEQ ID NO: 27) | |
| DE41 | 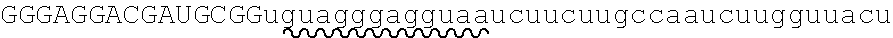 | 1 |
| | cgugTAGACGACUCGCUGAGGAUCCGAGA (SEQ ID NO: 28) | |
| DE44A | GGGAGGACGAUGCGGuggagaggggaaucguccugcgcacucugucuccug | 1 |
| | agagCAGACGACUCGCUGAGGAUCCGAGA (SEQ ID NO: 29) | |
| DE45 | GGGAGGACGAUGCGGuguauccgccaguauganuaacaucuauaagucccua | 1 |
| | uguCAGACGACUCGCUGAGGAUCCGAGA (SEQ ID NO: 30) | |
| DE46 | GGGAGGACGAUGCGGcuaaccuugunagggccccauacagcaucgagugacg | 1 |
| | gauCAGACGACUCGCUGAGGAUCCGAGA (SEQ ID NO: 31) | |
| DE47 | GGGAGGACGAUGCGGugcacaggaggugguacacugcgcucgauucaucag | 1 |
| | cgcaCAGACGACUCGCUGAGGAUCCGAGA (SEQ ID NO: 32) | |
| DE48 | GGGAGGACGAUGCGGcaugugagggaggagguccgcgucauaaacuccagg | 2 |
| | accaCAGACGACUCGCUGAGGAUCCGAGA (SEQ ID NO: 33) | |
| DE50 | GGGAGGACGAUGCGGaagcaauagcucgccguacaguuguccugccguucg | 1 |
| | uguuCAGACGACUCGCUGAGGAUCCGAGA (SEQ ID NO: 34) | |
| DE52 | 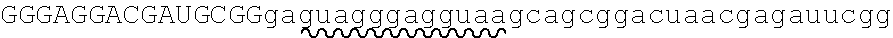 | 1 |
| | ugagCAGACGACUCGCUGAGGAUCCGAGA (SEQ ID NO: 35) | |
| 2DE8 | GGGAGGACGAUGCGGcgagcaaccggaacucggcuanuaugaccagccaacu | 1 |
| | uaaCAGACGACUCGCUGAGGAUCCGAGA (SEQ ID NO: 36) | |
| 2DE8A | GGGAGGACGAUGCGGcgagcaaccugaacucggcuauuaggaccagccaac | 3 |
| | uuaaCAGACGACUCGCUGAGGAUCCGAGA (SEQ ID NO: 37) | |
| 2DE11 | GGGAGGACGAUGCGGgaucggaaccagcgugacgaagcgcggaucaacuccg | 3 |
| | gugCAGACGACUCGCUGAGGAUCCGAGA (SEQ ID NO: 38) | |
| 2DE11A | GGGAGGACGAUGCGGgaucggaaccagcgugacgaagcgcggaucaacuccg | 1 |
| | gugCUGACGACUCGCUGAGGAUCCGAGA (SEQ ID NO: 39) | |
| 2DE12 | GGGAGGACGAUGCGGccgauuagaacugggucgcgcuguacccuagggauc | 1 |
| | gaCAGACGACUCGCUGAGGAUCCGAGA (SEQ ID NO: 40) | |
| OX1 | GGGAGGACGAUGCGGagacccaagcgccacgucuggcaugugagggaggag | 1 |
| | guacCAGACGACUCGCUGAGGAUCCGAGA (SEQ ID NO: 41) | |
| OX2 | GGGAGGACGAUGCGGagagccaagcgccacgucuggcaugugaggggggag | 1 |
| | guacCAGACGACUCGCUGAGGAUCCGAGA (SEQ ID NO: 42) | |
| OX3B | GGGAGGACGAUGCGGaaacucaucgguagccuuccugcggucagucuauua | 1 |
| | ggacCAGACGACUCGCUGAGGAUCCGAGA (SEQ ID NO: 43) | |
| OX4B | GGGAGGACGAUGCGGcaanuaccucagccucccuagacacgucgucuauua | 1 |
| | ggacCAGACGACUCGCUGAGGAUCCGAGA (SEQ ID NO: 44) | |
| OX5A | GGGAGGACGAUGCGGcagucuuccgguaagcacggaggugaggggagcuua | 1 |
| | gcguCAGACGACUCGCUGAGGAUCCGAGA (SEQ ID NO: 45) | |
| OX6 | GGGAGGACGAUGCGGauaugccaugggucgcucgagugaggucgucuauu | 1 |
| | aggacCAGACGACUCGCUGAGGAUCCGAGA (SEQ ID NO: 46) | |
| OX7B | GGGAGGACGAUGCGGagagccaagcgccacgucuggcaugugagggaggag | 3 |
| | guacCAGACGACUCGCUGAGGAUCCGAGA (SEQ ID NO: 47) | |
| OX8 | GGGAGGACGAUGCGGauuggcgcuauuaggaccagcuccguccgcaacugg | 2 |
| | ucccGAGACGACUCGCUGAGGAUCCGAGA (SEQ ID NO: 48) | |
| OX9 | GGGAGGACGAUGCGGgaacagacccauggcaaucucgcgacgucuucggccg | 1 |
| | cugCAGACGACUCGCUGAGGAUCCGAGA (SEQ ID NO: 49) | |
| OX10 | GGGAGGACGAUGCGGuacaacagguucauacggcgcguuguuccuuggcug | 1 |
| | acgCAGACGACUCGCUGAGGAUCCGAGA (SEQ ID NO: 50) | |
| OX11 | GGGAGGACGAUGCGGcacuauuaggaccagugccuguugucucgauaagcu | 2 |
| | ccgcCAGACGACUCGCUGAGGAUCCGAGA (SEQ ID NO: 51) | |
| OX12 | GGGAGGACGAUGCGGauuggcgcuauuaggaccagcuccguccgcaacuga | 1 |
| | ucccGAGACGACUCGCUGAGGAUCCGAGA (SEQ ID NO: 52) | |
| OX13A | GGGAGGACGAUGCGGcuauuaggaccagccguguagaauucguagcgaug | 1 |
| | ugacgCAGACGACUCGCUGAGGAUCCGAGA (SEQ ID NO: 53) | |
| OX13B | GGGAGGACGAUGCGGuucgcgcuauuaggaccagugcgaacguggguaua | 1 |
| | cauguCAGACGACUCGCUGAGGAUCCGAGA (SEQ ID NO: 54) | |
| 2OX2B | GGGAGGACGAUGCGGaacacacgggacgagccuggcgguugucgucuauua | 1 |
| | ggacCAGACGACUCGCUGAGGAUCCGAGA (SEQ ID NO: 55) | |
| 2OX3 | GGGAGGACGAUGCGGguccaugcuuuaaacugcaauuucccgunuacacgg | 2 |
| | gcuguCAGACGACUCGCUGAGGAUCCGAGA (SEQ ID NO: 56) | |
| 2OX3M | GGGAGGACGAUGCGGaccaccgaaucacgaggugcgagacauugguuccccg | 1 |
| | ccgCAGACGACUCGCUGAGGAUCCGAGA (SEQ ID NO: 57) | |
| 2OX4 | GGGAGGACGAUGCGGgggacaauaguccacgacuacaugucggugcgucgg | 1 |
| | agguCAGACGACUCGCUGAGGAUCCGAGA (SEQ ID NO: 58) | |
| 2OX6A | GGGAGGACGAUGCGGcuauuaggaccagcugccaaugumagucuacccca | 1 |
| | gcagCAGACGACUCGCUGAGGAUCCGAGA (SEQ ID NO: 59) | |
| 2OX6C | GGGAGGACGAUGCGGcuuacguauggucacggaggugugggggaacauaca | 1 |
| | gcagCAGACGACUCGCUGAGGAUCCGAGA (SEQ ID NO: 60) | |
| 2OX8 | GGGAGGACGAUGCGGuuggugaccuauucaggcguaggcauauaaacuacg | 1 |
| | aggcCAGACGACUCGCUGAGGAUCCGAGA (SEQ ID NO: 61) | |
| 2OX9 | GGGAGGACGAUGCGGcuauuaggaccagcugccaaugumagucuacccca | 1 |
| | gcggCAGACGACUCGCUGAGGAUCCGAGA (SEQ ID NO: 62) | |
| 2OX11 | GGGAGGACGAUGCGGgcacgacacgccgauuagaacugggcgaucuugguc | 1 |
| | gagcCAGACGACUCGCUGAGGAUCCGAGA (SEQ ID NO: 63) | |
| 2OX12 | GGGAGGACGAUGCGGcgauacgaccgcaugaguauaccgucgugcuucccgg | 1 |
| | cugCAGACGACUCGCUGAGGAUCCGAGA (SEQ ID NO: 64) | |
| 2OX13 | GGGAGGACGAUGCGGauuggcgcuauuaggaccagcuccguccgcaaccgg | 1 |
| | ucccCAGACGACUCGCUGAGGAUCCGAGA (SEQ ID NO: 65) | |
| 2OX14 | GGGAGGACGAUGCGGauuggcgcuauuaggaccagcuccguccgcaacugg | 1 |
| | ucccCAGACGACUCGCUGAGGAUCCGAGA (SEQ ID NO: 66) | |

Cloning, Sequencing and RNA Preparation:

At round 11 and following the final round of selection, cDNA was removed for cloning and sequencing of individual aptamers. Sequencing was carried out at the Johns Hopkins Genetic Resources Core Facility. Large quantities of aptamer for further analysis were generated from clone DNA by transcription using the Durascribe T7 kit (Epicentre, Madison, Wis.). RNA was recovered with the RNA Clean and Concentrator-5 kit (Zymo Research, Irvine, Calif.), eluted in H₂O and further concentrated by vacuum when necessary. Consensus sequences were identified using a ClustalW multiple sequence alignment (Higgins et al., 1996). Secondary structures were generated with Mfold software (Zuker, 2003).

Binding Assays: Binding assays were performed with individual aptamers, DE3A and OX3B, and control aptamers 1 and 2. RNA was dephosphorylated with bacterial alkaline phosphatase (Invitrogen, Grand Island, N.Y.) and 5′ end-labeled with γ-³²P-ATP (Perkin Elmer, Waltham, Mass.) using T4 polynucleotide kinase (New England Biolabs, Ipswich, Mass.). RNA was diluted to 2,000 cpm/μl and heated at 80° C. for 1 minute. For the assays targeting oxyHbS, the hemoglobin was thawed and used directly as in the SELEX process. The assays targeting deoxyHbS used fluorometHbS (FmetHbS) converted to the deoxygenated conformation as the target protein. FmetHbS was prepared following the procedure of Jayaraman et.al, (1993) using potassium hexacyanoferrate (III) (Sigma, St. Louis, Mo.) at a 10% excess, with dialysis in 0.2 M sodium phosphate buffer, pH 6.8, (Antonini & Brunori, 1971) followed by the conditions described by Jayaraman et al. (1993) Sodium Fluoride (Sigma, St. Louis, Mo.) was added at a 2:1 molar excess. Final buffer exchange into 2 mM HEPES, pH 7.4 was achieved with an Amicon Ultra 30K centrifugal filter tube (Millipore, Billerica, Mass.). Each protein dilution was in 15 μl of high-salt binding buffer. The FmetHbS dilution series also contained a 15× molar excess of inositol hexaphosphate (IHP, Sigma, St. Louis, Mo.), an allosteric effector that stabilizes the T quatemary structure (Jayaraman et al, 1993; Yohe et al, 2000). Five μl of labeled RNA were added to each tube of the dilution series, incubated at 37° C. for 15 minutes, and passed over a nitrocellulose membrane, with the unbound RNA captured on a nylon membrane. The percentage of RNA bound to protein was calculated for each dilution.

Oxyhemoglobin Dissociation Curves: An aliquot of whole blood was spun and the plasma removed. A volume of water alone or water containing 300 μg of aptamer, equivalent to 4-fold the volume of the whole blood aliquot, was added to the cell pellet and mixed to lyse the cells. The aptamer:heme molar ratio was 1:10. This preparation was loaded onto a Hemox Analyzer (TCS Scientific Corporation, New Hope, Pa.) to generate oxyhemoglobin dissociation curves and obtain p50 values.

Polymerization Assays: Sickle hemoglobin in 1 M potassium phosphate buffer pH 7.1 was thawed on ice, concentrated in an Amicon Ultra 30K centrifugal filter tube (Millipore, Billerica, Mass.) at 4° C. and kept on ice. Aptamers in distilled H₂O, or H₂O alone as a control, were thawed on ice, denatured at 80° C. for 1 minute, allowed to cool to room temperature, and placed on ice. Control 1 and Control 2, unrelated aptamers possessing flanking sequences and nucleotide modifications identical to those generated here, were used as negative aptamer controls. Sodium dithionite was employed at a 4:1 dithionite:heme molar excess in order to ensure maximal deoxygenation of HbS. It was prepared in deoxygenated potassium phosphate buffer, thereby preventing the generation of reactive oxygen byproducts (Di Iorio, 1981). To deoxygenate sodium dithionite powder, it was placed in a tube with a rubber septum cap and flushed with nitrogen gas (Adachi & Asakura, 1979). Potassium phosphate buffer was deoxygenated similarly in a separate tube. Sodium dithionite stock solution and subsequent dilutions were then made using a Hamilton gas-tight syringe to transfer buffer from one tube to another and kept on ice (Adachi & Asakura, 1979). All components were added on ice to a closed quartz cuvette (Starna Cells, Atascadero, Calif.) that had been flushed with nitrogen gas, and the temperature increased to 37° C. The final concentrations of all components in the cuvette were 0.12 mM HbS (heme), 0.48 mM sodium dithionite, and 0.012 mM aptamer in 1.45-1.55 M potassium phosphate, pH 7.42-7.80. Buffer concentration and pH were consistent within each experiment. Turbidity was measured at regular intervals with a Beckman DU-640B spectrophotometer (Beckman Coulter, Inc., Brea, Calif.) at a wavelength of 700 nm (Adachi & Asakura, 1979; Knee & Mukerji, 2009; Moffat & Gibson, 1974). Measurements were also taken at 540, 560 and 576 nm in order to determine the proportions of hemoglobin derivatives present. For concentration-response assays, only the final concentration of aptamer was varied. For experiments involving HbF, HbF dialyzed in H₂O was added in increasing concentrations, keeping the total volume and final concentration of HbS constant.

Electron microscopy: At the 78-minute time point during the polymerization assay, where the H₂O control was approaching maximal polymerization and the solutions with aptamer had just begun to polymerize, 20 μl was removed from each solution with a Hamilton syringe and added to 1 ml of deoxygenated 2% glutaraldehyde. Samples were spun at 7000 g for 10 minutes and brought to 30 μl to concentrate fibers. One microliter of each sample was adsorbed to a glow discharged carbon coated 400 mesh copper grid (Electron Microscopy Sciences, Hatfield, Pa.) by floatation for 2 minutes. Grids were quickly blotted then rinsed in 3 drops (1 minute each) of tris-buffered saline. Grids were negatively stained in 2 consecutive drops of 0.75% uranyl formate, blotted, then quickly aspirated to get a thin layer of stain covering the sample. Grids were imaged on a Phillips CM-120 transmission electron microscope operating at 80 kV. Images were taken with an 8 megapixel CCD camera (Advanced Microscopy Techniques, Wobum, Mass.).

In vitro Erythrocyte Sickling Assay: Lipofection was employed to facilitate the uptake of aptamer by RBCs. In one tube, 1.4 μl of Lipofectamine 3000 (Invitrogen, Carlsbad, Calif.) was added to 4.4 μl of Opti-MEM media. Separately, 275 μg aptamer in 5 μl of H₂O were denatured at 80° C. for 1 minute. To this, 0.2 μl of P3000 reagent was added. These two mixtures were combined and incubated at room temperature for 10 minutes. Following incubation, 10 μl of erythrocytes, washed and suspended in Opti-MEM at a packed cell volume of 25%, were mixed into the Lipofectamine mixture and incubated at 37° C. for 22 hours. Lipofections were then diluted with 700 μl PBS and sickling assays were performed immediately.

For each lipofection, an 85 μl aliquot of cells was transferred to a septum cap cuvette, which was then flushed with an atmosphere of 96% nitrogen/4% oxygen (Airgas East, Berwyn, Pa.). (Safo et al., 2004) Cuvettes were incubated at 37° C. for 1 hour, followed by fixation with 10 μl of 25% glutaraldehyde. The fixed cells were transferred to a microscope slide for visual enumeration of sickle forms.

### Results

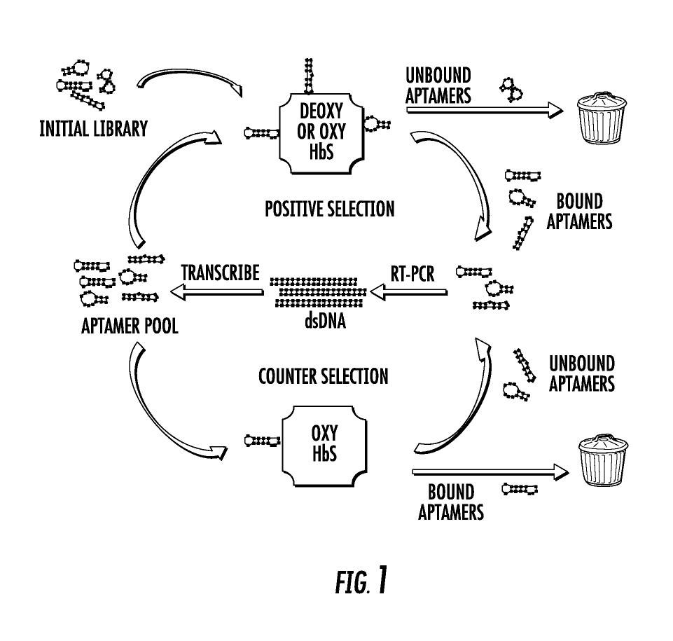

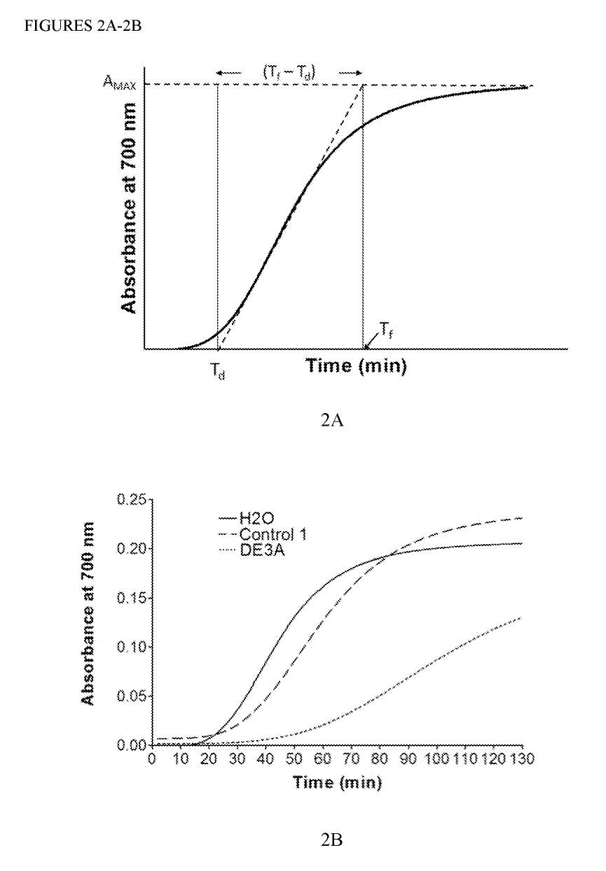

Aptamer pools selected using oxyHbS and deoxyHbS are predominately distinct: Our goal was to select for one or more aptamers that, when bound to HbS, would inhibit polymerization under hypoxic conditions. To preclude any false assumptions regarding which hemoglobin structure may prove to be the best target for an effective aptamer, two separate aptamer pools were generated: one against deoxyHbS and one against oxyHbS. The selection scheme is shown in FIG. 1 and described in detail in “Materials and Methods.”

Individual aptamers were sequenced by cloning the cDNA at rounds 11 and following the final rounds of selection. A total of 92 clones were sequenced, with 60 unique sequences represented (Table 1). Thirty-four unique aptamers were generated in the deoxyHbS-binding selection and 26 in the oxyHbS-binding selection. There were two consensus sequences specific to the deoxyHbS-targeting pool, and two specific to the oxyHbS-targeting pool, with the exception of one aptamer in each pool that contained a consensus sequence found in the other pool. There was only one aptamer whose entire sequence was common to both selections; thus, the aptamers selected against deoxygenated HbS are predominately distinct from those selected against oxygenated HbS.

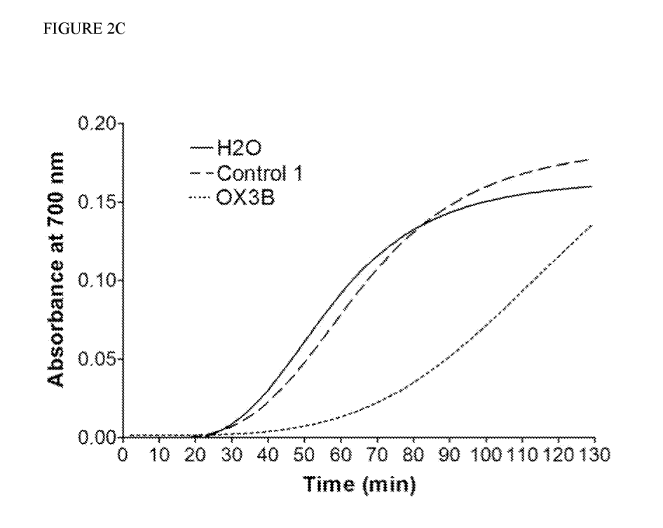

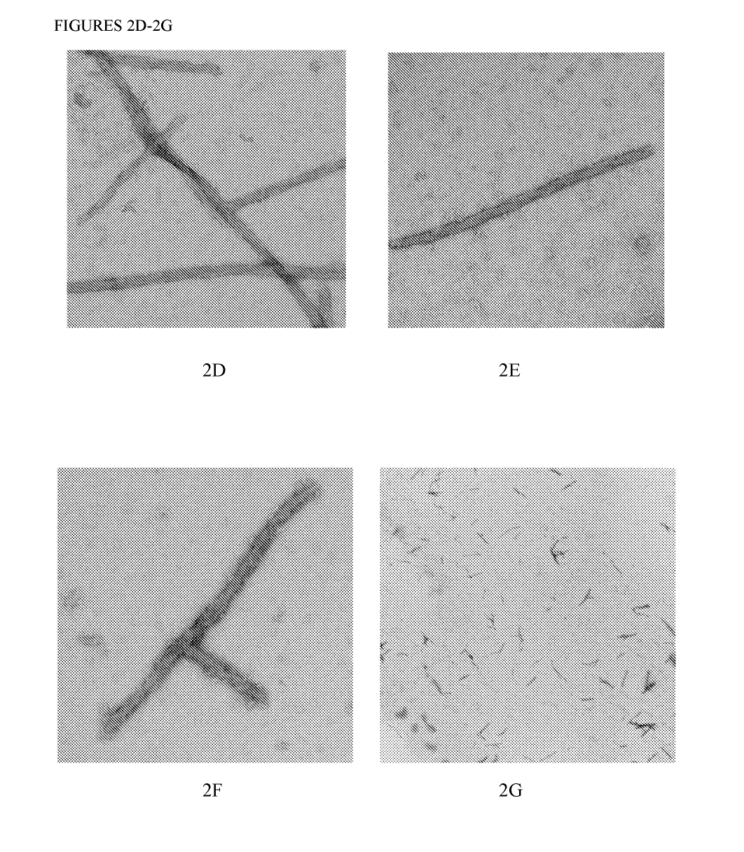

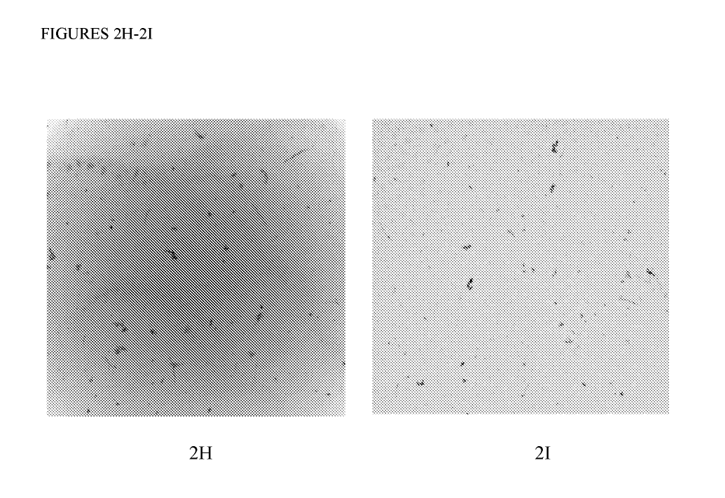

Identification of individual aptamers that inhibit polymerization of deoxygenated sickle hemoglobin: Individual aptamers were amplified and evaluated for their ability to inhibit polymerization of deoxyHbS in an oxygen-depleted solution. In this system, the addition of dithionite consistently resulted in solutions of 90-94% deoxyHbS. A typical polymerization tracing from this type of assay is shown in FIG. 2A. The delay time, T_{d}, reflects the time required for the formation of nuclei (Adachi & Asakura, 1979). In order to show that our result was not simply due to protein salting out in high concentration phosphate buffer, electron microscopy was employed to visualize the reaction products at the 78-minute time point. We confirmed that sickle hemoglobin fibers were present in each sample, and that fibers were branching in a heterogeneous manner (FIG. 2D, FIG. 2E, and FIG. 2F). A random inspection of the entire surface of the grids established that the fibers were generally equally distributed across each grid, and that fibers were present in a much greater quantity in the H₂O control than in the samples containing aptamer (FIG. 2G, FIG. 2H, and FIG. 2I). No further quantitation was done, as the process of staining for electron microscopy breaks the fibers (Briehl et al, 1990). This result is consistent with the findings of Wang et al (2000), who found that fibers formed in 1.5M phosphate buffer with the same structures as those formed in 0.5 M phosphate buffer.

Two aptamers were found that consistently and significantly inhibited polymerization: DE3A, generated from the selection targeting deoxyHbS, and OX3B, generated from the selection targeting oxyHbS. Delay times and slopes of polymerization in the presence of either DE3A or OX3B were compared to those obtained in the presence of unrelated aptamer control 1 or with no aptamer present (water control) (FIG. 2B and FIG. 2C). The delay time in the presence of either aptamer was significantly longer compared to the controls.

Additionally, both aptamers significantly reduced the slopes of the polymerization curve during exponential growth, as compared to either control 1 or water alone. These results indicate that both DE3A and OX3B inhibit the kinetics of HbS polymerization by extending the time required for nucleation and slowing the rate of polymerization.

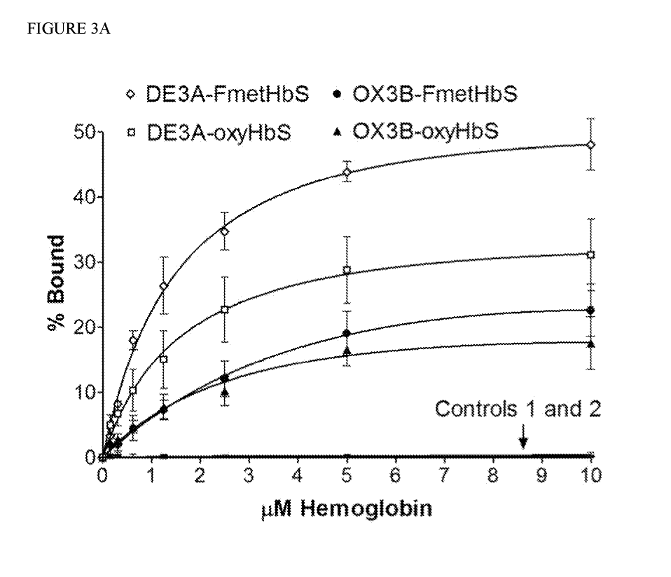

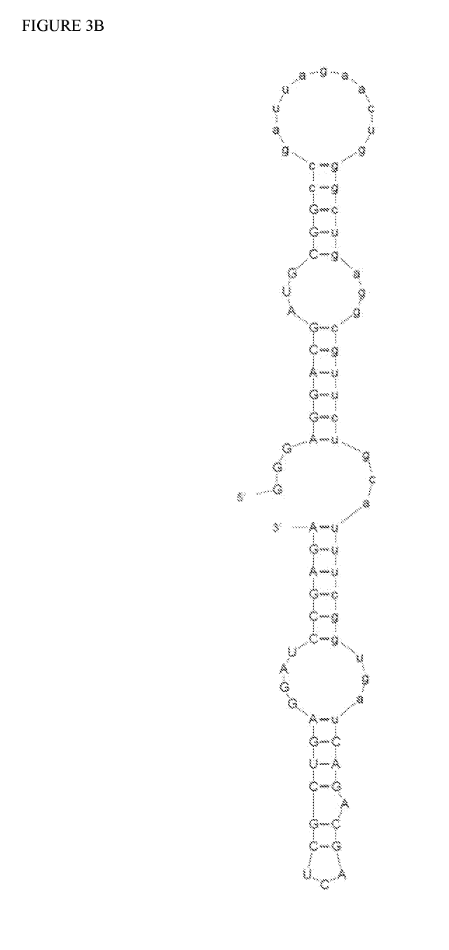

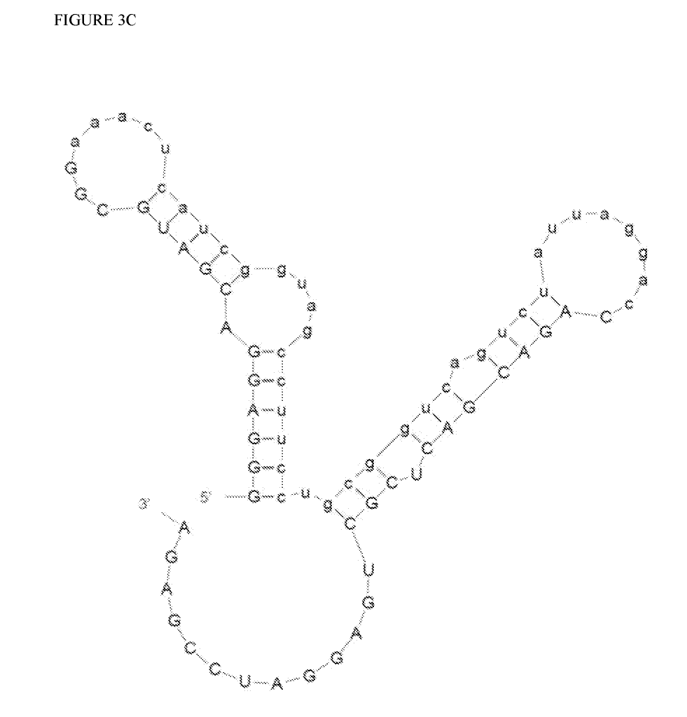

Aptamers DE3A and OX3B bind HbS: Aptamers DE3A, OX3B, and control aptamers 1 and 2 were assayed for their binding affinities to both oxyHbS and deoxyHbS (FIG. 3A).

In these assays, FmetHbS was used in place of deoxyHbS. FmetHbS is advantageous because, with addition of IHP, the content of T-state hemoglobin is 72% or more (Jayaraman et al, 1993) in the non-oxygen-depleted environment of the binding assay, as compared to 18-28% for vacuum-deoxygenated HbS. DE3A showed nearly identical binding affinities to FmetHbS and oxyHbS, with respective K_{d} values of 1.68 μM and 1.74 μM. OX3B bound to oxyHbS with a K_{d} of 3.56 μM and to FmetHbS with a K_{d} of 8.57 μM. These K_{d} values indicate that each aptamer maintains its affinity for HbS as the protein changes conformation, although OX3B may exhibit a slightly higher affinity for oxyHbS. There was no binding between either the control aptamer or hemoglobin, in either conformation. Aptamer variable region sequences are shown in Table 2, and aptamer secondary structures are shown in FIG. 3B and FIG. 3C.

TABLE 2
Sequences of aptamer variable regions. Flanking
sequences are 5′-GGGAGGACGAUGCGG(N40)CAGACGACUCGCU
GAGGAUCCGAGA-3′, where (N40) is the variable
region shown. (SEQ ID NO: 5 is the 5′ flanking
region (5′-GGGAGGACGAUGCGG-3′) and SEQ ID NO: 6 is
the 3′ flanking region (5′-CAGACGACUCGCUGAGGAUCCGA
GA-3′)).

| Aptamer | Sequence of 40 nt variable region |
|---|---|
| DE3A | CCGAUUAGAACUGGGCUGAGGCGUUCUGCAUUUCGGUG |
| | AU (SEQ ID NO: 1) |
| OX3B | AAACUCAUCGGUAGCCUUCCUGCGGUCAGUCUAUUAGG |
| | AC (SEQ ID NO: 2) |
| Control | AGCGACUGACGAUCUUGAGUAAACCGCUCAUCCACGUA |
| 1 | GU (SEQ ID NO: 3) |
| Control | UCACCAGCGCUCUACGAACCCCGCAUUCCCAGUUGCUA |
| 2 | CA (SEQ ID NO: 4) |

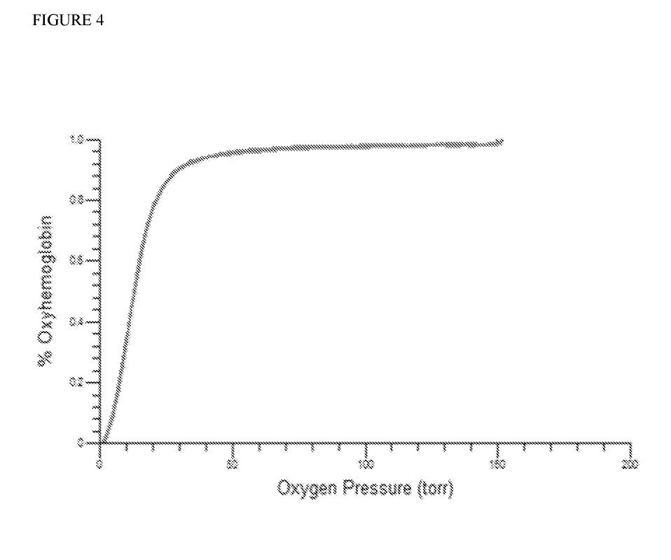

Aptamer binding does not alter the oxygen affinity of hemoglobin: Hemolysates from SCD patients were utilized to determine whether the binding of aptamer to HbS had any detectable effect on the affinity of HbS for oxygen (FIG. 4). Analysis of lysate yielded lower p50 values than whole cell analysis (Hirsch et al, 1993), as reflected in the average control p50 value of 15.51 mm Hg. The resulting ODC of both aptamers and the no-aptamer control were indistinguishable; p50 values were not significantly different.

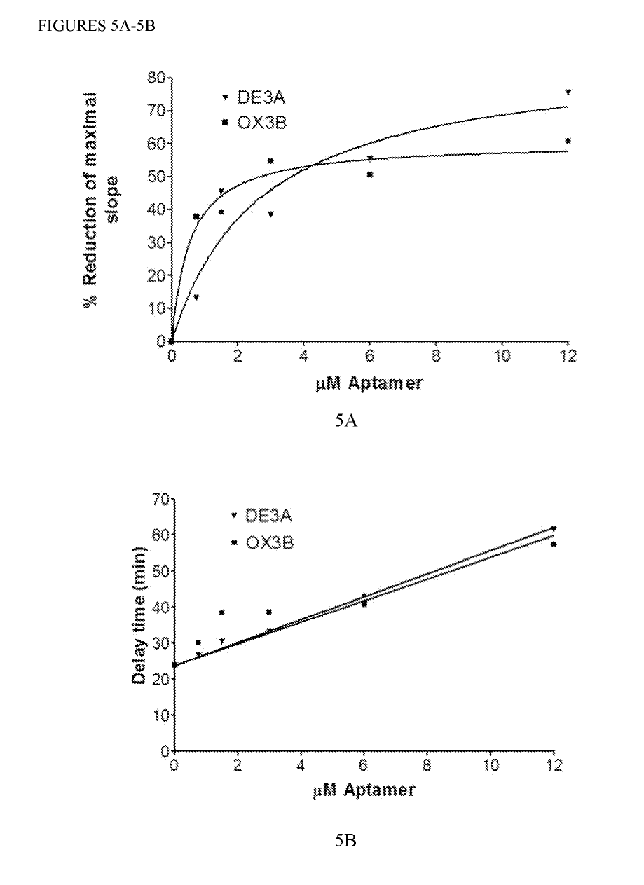

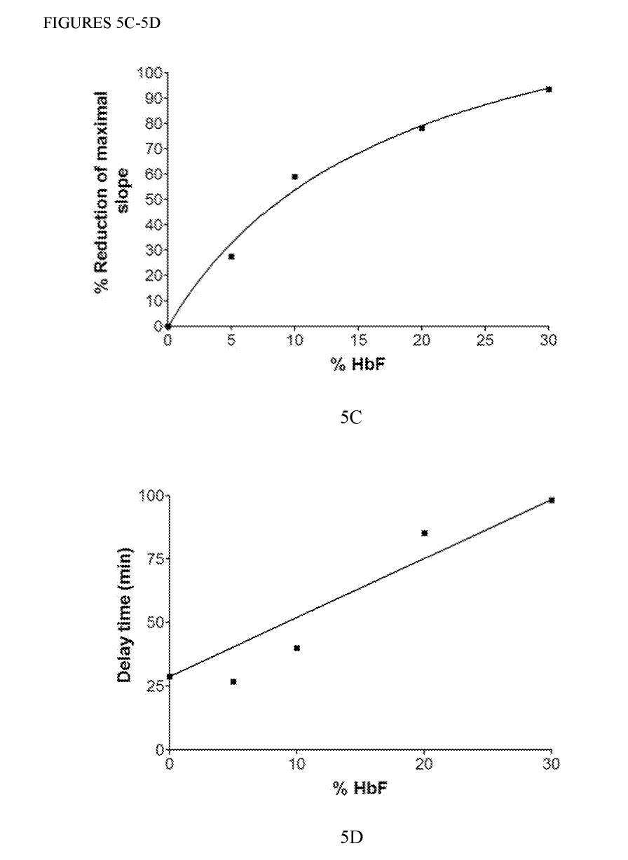

Concentration response of delay times and polymerization rates: The relationship between aptamer concentration and rate of polymerization is shown in FIG. 5A. At an aptamer concentration of 12 μM (an aptamer:heme ratio of 1:10), the rate of polymerization was inhibited 75.4% with DE3A and 60.8% with OX3B. Although DE3A caused a higher maximal level of inhibition than OX3B, at aptamer concentrations below approximately 4 μM, OX3B appeared to be more effective than DE3A in inhibiting the polymerization rate.

The increase in delay time of polymerization with increasing aptamer concentration is shown in FIG. 5B. The delay time increased 2.6-fold with 12 μM DE3A, and 2.4-fold with 12 μM OX3B. In contrast to the aptamers' effect on polymerization rate, where nearly maximal inhibition was reached at an aptamer:heme ratio of 1:10, extrapolation of the delay time curves suggests that increasing the relative aptamer concentrations could further extend the delay time.

To determine whether this degree of inhibition is likely to have physiologic significance, we compared the inhibition of polymerization by the aptamer to that of HbF in polymerization assays. With a mixture containing 10% HbF, 50% inhibition of polymerization was achieved with almost complete inhibition at 30% HbF (FIG. 5C). In comparison, approximately 50% inhibition of polymerization was seen with either aptamer at a concentration of 4 μM; at 3 times this concentration, approximately 60% and 75% inhibition was seen with OX3B and DE3A, respectively. The delay time with 12 μM aptamer was approximately 2.5 times greater than the delay time without aptamer. A similar extension of the delay time was also seen with the addition of approximately 15-20% HbF (FIG. 5D). This suggests that the aptamers could inhibit the rate of polymerization of HbS at the same order of magnitude as HbF, but higher concentrations and ratios of aptamer may be necessary for maximal effect.

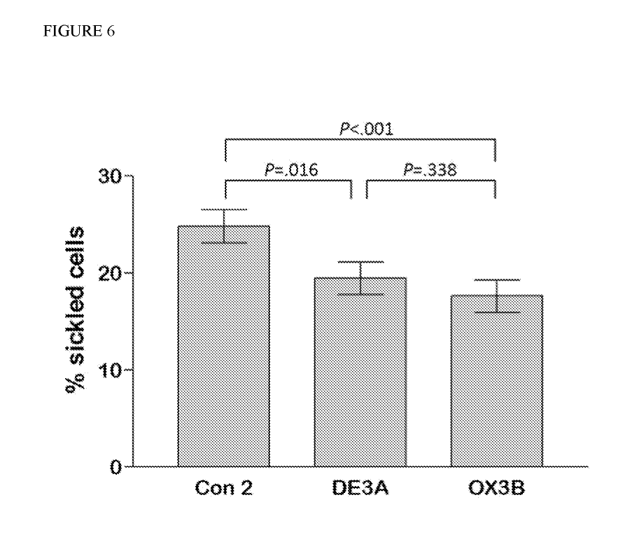

Aptamers reduce sickling of HbSS erythrocytes in vitro: Aptamers DE3A and OX3B were tested to determine their effect on HbSS erythrocyte sickling under hypoxic conditions. Lipofection was carried out with a quantity of aptamer such that, assuming aptamer equilibration between the extracellular and intracellular compartments, the final intracellular concentration would be 0.5 mM, for an aptamer:heme ratio of 1:10, the same as that applied in the polymerization assays. Internalized aptamer was not quantified following lipofection, so it is possible that the intracellular concentration was less than expected. Following lipofection, both aptamers significantly inhibited sickling as compared to control 2 (FIG. 6). Aptamer DE3A reduced the number of sickled cells by 21.5% while OX3B reduced the number by 29.0%. There was no significant difference between the control aptamer and water when compared in the same assay (data not shown). Binding data in FIG. 3A indicate that control 2 does not bind to HbS.

### Discussion

The ability to block the polymerization of HbS without affecting hemoglobin function or creating unwanted side effects should be curative. The compositions of the present invention provide one or more aptamers which, when bound to sickle hemoglobin, that inhibit the polymerization of HbS and thereby reduce the sickling of HbS-containing erythrocytes under hypoxic conditions. At least two such aptamers, DE3A and OX3B, have been disclosed herein. Each aptamer slows homogeneous nucleation, as the delay times with an aptamer:heme ratio of 1:10 were extended 2.6-fold with DE3A and 2.4-fold with OX3B. Additionally, once polymerization was initiated, both significantly slowed the rate of polymer formation.

Variation in delay time has been associated with the severity of disease (Hofrichter et al, 1974; Eaton et al, 1976; Du et al, 2015). Sickle cells can occlude capillary beds, where blood flow is most restricted, or in the venules where adhesion to endothelial cells presents a further opportunity for occlusion (Kaul et al, 1995; Kaul et al, 2009). Even partial inhibition of polymerization can have important therapeutic effects; the longer the delay of polymerization, the more likely it is that the erythrocyte will pass through the capillaries and venules. Polymerization delay times within erythrocytes are very sensitive to physiological conditions and thus vary from patient to patient (Du et al, 2015; Coletta et al, 1982; Zarkowsky and Hochmuth, 1975), potentially leading to variability in disease manifestation among individuals with SCD. An agent such as those disclosed herein, with the ability to extend delay times can shift the balance toward a less severe disease course. Our invention suggests that, if delivered intracellularly at high concentrations, the inventive aptamers can extend the delay time and further slow the rate of growing polymers enough to allow a therapeutically significant proportion of cells to escape occlusion.

Achieving preparations consisting entirely of deoxyHS presented challenges.

Ultimately, the efficacy of individual aptamers was evaluated based on each aptamer's ability to inhibit polymerization, regardless of the origin of its selection. Each aptamer (DE3A and OX3B) binds to both oxygenated and deoxygenated hemoglobin with similar affinities. This suggests that they each bind to structures that do not change during the conformational shift between R- and T-state. This can be a beneficial quality in vivo, as the aptamer, once bound, would remain bound rather than undergoing detachment at each conformational shift. We examined whether the binding of aptamer to hemoglobin had any effect on the ODC (FIG. 4). A right-shift of the curve, reflecting a reduced affinity for oxygen, could be detrimental to the patient with SCD, as it would result in an increase in deoxygenated molecules. A left-shift of the ODC, while potentially protective due to an increase in the proportion of oxyHbS, can lead to impaired oxygen delivery to the tissues and erythrocytosis (Messmore & Choudhury, 1993). We found that the binding of neither DE3A nor OX3B of the present invention, perturb hemoglobin's oxygen affinity in vitro.

When DE3A and OX3B were introduced into HbS-containing erythrocytes and exposed to hypoxic conditions for one hour, they reduced the percentage of sickled cells by 21.5% and 29.0%, respectively. We did not quantify the amount of aptamer within the erythrocytes following lipofection; however, we used a concentration of 0.5 mM aptamer in these studies, so if complete equilibration occurred, an aptamer:heme ratio of 1:10 would have resulted. Previous studies have shown that erythrocytes are difficult to transfect (Maurisse et al, 2010). A study in which lipofectamine was employed to transfect erythrocytes resulted in a transfection efficiency of 40-50% (Chen et al, 2008). It is likely that our transfection efficiency was below 100%, with the actual concentration of aptamer within lipofected cells well below 0.5 mM. It is promising, then, that an aptamer:heme ratio potentially much less than 1:10 could reduce sickling by approximately 30%, or that perhaps a greater inhibition could be achieved with higher relative concentrations.

The present inventors show that, as an estimate, half-maximal inhibition of the rate of polymerization of HbS by the inventive aptamers would produce an effect roughly equivalent to 10% HbF, greater than the average increase in HbF (3.6%) or final level of HbF (8.6%) achieved with hydroxyurea in the MSH trial (Charache et al, 1996). Higher concentrations could potentially cause greater effects. This presupposes that delivery of the aptamers into circulating erythrocytes could be achieved, a hurdle which will need to be addressed. Given the high molar ratios required to see effects on polymerization of HbS, our current aptamers would most likely need to be concentrated in erythrocytes to be useful in vivo; given that the aptamers bind to hemoglobin, this is possible.

An important benefit of employing aptamers as therapeutic agents is that they are easily modified. Our aptamers incorporate 2′-fluoro nucleotides to protect against nuclease degradation (Sundaram et al, 2013). Macugen, an aptamer that has been approved by the U.S. Food and Drug Administration for the treatment of age-related macular degeneration, is an example of an effective, similarly modified aptamer (Lee et al, 2005; Ng & Adamis, 2006). Further modification of the nucleotide sequence or creation of a multivalent aptamer could improve binding affinities, thereby potentially increasing the ability to inhibit polymerization (Nonaka et al, 2013; Ahmad et al, 2012). Alternatively, it may be possible to truncate our existing aptamers while retaining inhibitory function, which might increase cellular uptake.

Without being limited to any particular theory, the present inventors can only speculate on the mechanism by which aptamers inhibit polymerization, but given that DE3A appears to affect both the rate and extent of HbS polymerization, whereas OX3B seems to inhibit only the rate of polymerization, DE3A may act by binding HbS monomers, whereas OX3B may simply inhibit polymerization of the elongating filaments. Neither aptamer appeared to induce nucleation, which might be a property of a species that could bind to the growing end of filaments and induce the filamentous conformation in monomers, as occurs with actin and its filamentous end-binding proteins (Casella et al, 1986).

In summary, the present inventors have created at least two aptamers with the ability to inhibit the polymerization of HbS in lysates and sickling in erythrocytes under hypoxic conditions. In addition, the fact that we were able to see inhibition of sickling in a cellular system suggests that the aptamers can bind and exert their effects under physiologic conditions. With the ability to deliver these aptamers intracellularly to erythrocytes, these two HbS polymerization-inhibiting RNA aptamers could potentially reduce or eliminate the consequences of sickling in patients with SCD.

All publications, patent applications, patents, and other references mentioned in the specification are indicative of the level of those skilled in the art to which the presently disclosed subject matter pertains. All publications, patent applications, patents, and other references (e.g., websites, databases, etc.) mentioned in the specification are herein incorporated by reference in their entirety to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

Adachi, K., Asakura, T. (1979) Nucleation-controlled aggregation of deoxyhemoglobin S.
Journal of Biological Chemistry, 254, 7765-7771.
Ahmad, K. M., Xiao, Y., Soh, H. T. (2012) Selection is more intelligent than design: improving the affinity of a bivalent ligand through directed evolution. Nucleic Acids Research, 40, 11777-11783.
Antonini, E., Brunori, M. (1971) Hemoglobin and Myoglobin in Their Reactions With Ligands. North-Holland Publishing Company, Amsterdam.
Ataga, K. I., Stocker, J. (2015) The trials and hopes for drug development in sickle cell disease. British Journal of Haematology, 170, 768-780.
Benesch, R. E., Benesch, R., Yung, S. (1973) Equations for the spectrophotometric analysis of hemoglobin mixtures. Analytical Biochemistry. 55, 245-248.
Briehl, R. W., Mann, E. S., Josephs, R. (1990) Length distributions of hemoglobin S fibers.
Journal of Molecular Biology, 211, 693-698.
Casella, J. F., Maack, D. J., Lin, S. (1986) Purification and initial characterization of a protein from skeletal muscle that caps the barbed ends of actin filaments. Journal of Biological Chemistry, 261, 10915-10921.
Charache, S., Barton, F. B., Moore, R. D., Terrin, M. L., Steinberg, M. H., Dover, G. J., Ballas, S. K., McMahon, R. P., Castro, O., Orringer, E. P., and investigators of the Multicenter Study of Hydroxyurea in Sickle Cell Anemia. (1996) Hydroxyurea and sickle cell anemia. Clinical utility of a myelosuppressive “switching” agent. The Multicenter Study of Hydroxyurea in Sickle Cell Anemia. Medicine (Baltimore), 75, 300-326.
Chen, S. Y., Wang, Y., Telen, M. J., Chi, J. T. (2008) The genomic analysis of erythrocyte microRNA expression in sickle cell diseases. PLoS One. 3, e2360.
Coletta, M., Hofrichter, J., Ferrone, F. A., Eaton, W. A. (1982) Kinetics of sickle haemoglobin polymerization in single red cells. Nature, 300, 194-197.
Di Iorio, E. E. (1981) Preparation of derivatives of ferrous and ferric hemoglobin. Methods in Enzymology, 76, 57-72.
Du, E., Diez-Silva, M., Kato, G. J., Dao, M., Suresh, S. (2015) Kinetics of sickle cell biorheology and implications for painful vasoocclusive crisis. Proceedings of the National Academy of Sciences of the United States of America, 112, 1422-1427.
Dykes, G., Crepeau, R. H., Edelstein, S. J. (1978) Three-dimensional reconstruction of the fibres of sickle cell haemoglobin. Nature, 272, 506-510.
Eaton, W. A., Hofrichter, J., Ross, P. D. (1976) Delay time of gelation: a possible determinant of clinical severity in sickle cell disease. Blood, 47, 621-627.
Ferrone, F. A., Hofrichter, J., Eaton, W. A. (1985) Kinetics of sickle hemoglobin polymerization. II. A double nucleation mechanism. Journal of Molecular Biology, 183, 611-631.
Higgins, D. G., Thompson, J. D., Gibson, T. J. (1996) Using CLUSTAL for multiple sequence alignments. Methods in Enzymology, 266, 383-402.
Hirsch, R. E., Lin, M. J., Pulakhandam, U. R., Nagel, R. L., Sandberg, S. (1993) Hemoglobin oxygen affinity is increased in erythropoietic protoporphyria. Photochemistry and Photobiology, 57, 885-888.
Hofrichter, J. (1986) Kinetics of sickle hemoglobin polymerization. III. Nucleation rates determined from stochastic fluctuations in polymerization progress curves. Journal of Molecular Biology, 189, 553-571.
Hofrichter, J., Ross, P. D., Eaton, W. A (1974) Kinetics and mechanism of deoxyhemoglobin S gelation: a new approach to understanding sickle cell disease. Proceedings of the National Academy of Sciences of the United States of America, 71, 4864-4868.
Jayaraman, V., Rodgers, K. R., Mukerji, I., Spiro, T. G. (1993) R and T states of fluoromethemoglobin probed by ultraviolet resonance Raman spectroscopy. Biochemistry, 32, 4547-4551.
Kaul, D. K., Fabry, M. E., Costantini, F., Rubin, E. M., Nagel, R. L. (1995) In vivo demonstration of red cell-endothelial interaction, sickling and altered microvascular response to oxygen in the sickle transgenic mouse. Journal of Clinical Investigation, 96, 2845-2853.
Kaul, D. K., Finnegan, E., Barabino, G. A. (2009) Sickle red cell-endothelium interactions.
Microcirculation, 16, 97-111.
King, A., Shenoy, S. (2015) Evidence-based focused review of the status of hematopoietic stem cell transplantation as treatment of sickle cell disease and thalassemia. Blood, 123, 3089-3095.
Knee, K. M., Mukerji, I. (2009) Real time monitoring of sickle cell hemoglobin fiber formation by UV resonance Raman spectroscopy. Biochemistry, 48, 9903-9911.
Lee, J.-H., Canny, M. D., De Erkenez, A., Krilleke, D., Ng, Y.-S., Shima, D. T., Pardi, A., Jucker, F. (2005) A therapeutic aptamer inhibits angiogenesis by specifically targeting the heparin binding domain of VEGF165. Proceedings of the National Academy of Sciences of the United States ofAmerica, 102, 18902-18907.
Maurisse, R., De Semir, D., Emamekhoo, H., Bedayat, B., Abdolmohammadi, A., Parsi, H., Gruenert, D. C. (2010) Comparative transfection of DNA into primary and transformed mammalian cells from different lineages. Bio Med Central Biotechnology, 10:9.
Messmore, H. L., Choudhury, A. M. (1993) Polycythemia. In: Bick R L, Bennett J M, Brynes, R K, et al, eds. Hematology: Clinical and Laboratory Practice. Vol 1. Mosby-Year Book Inc., St. Louis, Mo.
Moffat, K., Gibson, Q. H. (1974) The rates of polymerization of sickle cell hemoglobin.
Biochemical and Biophysical Research Communications, 61, 237-242.
Nagel, R. L., Bookchin, R. M., Johnson, J., Labie, D., Wajcman, H., Isaac-Sodeye, W. A., Honig, G. R., Schiliro, G., Crookston, J. H., Matsutomo, K. (1979) Structural bases of the inhibitory effects of hemoglobin F and hemoglobin A₂ on the polymerization of hemoglobin S. Proceedings of the National Academy of Sciences of the United States of America, 76, 670-672.
Ng, E. W. M., Adamis, A. P. (2006) Anti-VEGF (Pegaptanib) therapy for ocular vascular diseases. Annals of the New York Academy of Sciences, 1082, 151-171.
Nonaka, Y., Yoshida, W., Abe, K., Ferri, S., Schulze, H., Bachmann, T. T., Ikebukuro, K. (2013) Affinity improvement of a VEGF aptamer by in silico maturation for a sensitive VEGF-detection system. Analytical Chemistry, 85, 1132-1137.
Perutz, M. F., Wilkinson, A. J., Paoli, M., Dodson, G. G. (1998) The stereochemical mechanism of the cooperative effects in hemoglobin revisited. Annual Review of Biophysics and Biomolecular Structure, 27, 1-34.
Que-Gewirth, N. S., Sullenger, B. A. (2007) Gene therapy progress and prospects: RNA aptamers. Gene Therapy, 14, 283-91.
Safo, M. K., Abdulmalik, O., Danso-Danquah, R. (2004) Structural basis for the potent antisickling effect of a novel class of five-membered heterocyclic aldehydic compounds. Journal of Medicinal Chemistry, 47, 4665-4676.
Serjeant, G. (1985) Sickle Cell Disease. Oxford University Press, New York, N. Y. Steinberg, M. H. (1999) Management of sickle cell disease. New England Journal of Medicine, 340, 1021-1030.
Sun, H., Zu, Y. (2015) A highlight of recent advances in aptamer technology and its application. Molecules, 20, 11959-11980.
Sundaram, P., Kurniawan, H., Byme, M. E., Wower, J. (2013) Therapeutic RNA aptamers in clinical trials. European Journal of Pharmaceutical Sciences, 48, 259-271.
Van Assendelft, O. W., Zijlstra, W. G. (1975) Extinction coefficients for use in equations for the spectrophotometric analysis of haemoglobin mixtures. Analytical Biochemistry, 69, 43-48.
Van Kampen, E. J., Zijlstra, W. G. (1983) Spectrophotometry of hemoglobin and hemoglobin derivatives. Advances in Clinical Chemistry, 23, 199-257.
Wang, Z., Kishchenko, G., Chen, Y., Josephs, R. (2000) Polymerization of deoxy-sickle cell hemoglobin in high-phosphate buffer. Journal of Structural Biology, 131, 197-209.
Yazdanbakhsh, K., Ware, R. E., Noizat-Pirenne, F. (2012) Red blood cell alloimmunization in sickle cell disease: pathophysiology, risk factors, and transfusion management. Blood, 120, 528-537.
Yohe, M. E., Sheffield, K. M., Mukerji, I. (2000) Solubility of fluoromethemoglobin S: effect of phosphate and temperature on polymerization. Biophysical Journal, 78, 3218-3226.
Zarkowsky, H. S., Hochmuth, R. M. (1975) Sickling times of individual erythrocytes at zero PO2. Journal of Clinical Investigation, 56, 1023-34.
Zuker, M. (2003) Mfold web server for nucleic acid folding and hybridization prediction. Nucleic Acids Research, 31, 3406-3415.

Although the foregoing subject matter has been described in some detail by way of illustration and example for purposes of clarity of understanding, it will be understood by those skilled in the art that certain changes and modifications can be practiced within the scope of the appended claims.

1. A method for inhibiting sickling of an erythrocyte, the method comprising introducing at least one polynucleotide aptamer into an erythrocyte comprising at least a first sickle hemoglobin (HbS) and a second HbS under conditions effective to specifically bind the at least one polynucleotide aptamer to the first HbS, wherein specifically binding the at least one polynucleotide aptamer to the first HbS inhibits polymerization of the first HbS with the second HbS, thereby inhibiting sickling of the erythrocyte.

2. The method of claim 1, wherein specifically binding the at least one polynucleotide aptamer to the first HbS inhibits polymerization of the first HbS with a second HbS without affecting the oxygen affinity of the first HbS.

3. (canceled)

4. The method of claim 1, wherein specifically binding the at least one polynucleotide aptamer to the first HbS occurs under hypoxic conditions.

5. The method of claim 1, wherein the at least one polynucleotide aptamer is an RNA aptamer.

6. The method of claim 1, wherein the first HbS and/or the second HbS is a monomer.

7. The method of claim 1, wherein the first HbS and/or the second HbS is a polymer.

8.-11. (canceled)

12. The method of claim 1, wherein the at least one polynucleotide aptamer specifically binds oxygenated HbS.

13. The method of claim 1, wherein the at least one polynucleotide aptamer specifically binds deoxygenated HbS.

14. The method of claim 1, wherein the at least one polynucleotide aptamer specifically binds both oxygenated HbS and deoxygenated HbS.

15. The method of claim 14, wherein the at least one polynucleotide aptamer specifically binds both oxygenated HbS and deoxygenated HbS with similar affinity.

16. The methods of claim 1, wherein specifically binding the at least one polynucleotide aptamer to the first HbS reduces the rate and extent of polymerization of the first HbS with the second HbS.

17. The method of claim 16, wherein the at least one polynucleotide aptamer comprises a nucleotide sequence selected from the group consisting of:
a) a nucleotide sequence at least 80% identical to SEQ ID NO:1;
b) a nucleotide sequence at least 90% identical to SEQ ID NO:1;
c) a nucleotide sequence at least 95% identical to SEQ ID NO:1;
d) a nucleotide sequence at least 99% identical to SEQ ID NO: 1;
e) the nucleotide sequence of SEQ ID NO: 1;
f) a nucleotide sequence at least 80% identical to SEQ ID NO:9;
g) a nucleotide sequence at least 90% identical to SEQ ID NO:9;
h) a nucleotide sequence at least 95% identical to SEQ ID NO:9;
i) a nucleotide sequence at least 99% identical to SEQ ID NO:9; and
j) the nucleotide sequence of SEQ ID NO:9.

18. The methods of claim 1, wherein specifically binding the at least one polynucleotide aptamer to the first HbS reduces the rate of polymerization without reducing the extent of polymerization of the first HbS with the second HbS.

19. The method of claim 18, wherein the at least one polynucleotide aptamer comprises a nucleotide sequence selected from the group consisting of:
a) a nucleotide sequence at least 80% identical to SEQ ID NO:2;
b) a nucleotide sequence at least 90% identical to SEQ ID NO:2;
c) a nucleotide sequence at least 95% identical to SEQ ID NO:2;
d) a nucleotide sequence at least 99% identical to SEQ ID NO:2;
e) the nucleotide sequence of SEQ ID NO:2;
f) a nucleotide sequence at least 80% identical to SEQ ID NO:43;
g) a nucleotide sequence at least 90% identical to SEQ ID NO:43;
h) a nucleotide sequence at least 95% identical to SEQ ID NO:43;
i) a nucleotide sequence at least 99% identical to SEQ ID NO:43; and
j) the nucleotide sequence of SEQ ID NO:43.

20. The method of claim 1, wherein the at least one polynucleotide aptamer is modified to prevent nuclease degradation.

21. The method of claim 1, wherein the at least one polynucleotide aptamer comprises at least one 2′-fluoro nucleotide.

22.-26. (canceled)


SEQUENCE LISTING (66 sequences)

SEQ ID NO: 1 - RNA, 40 nt, Artificial Sequence
  RNA oligonucleotide
ccgauuagaacugggcugaggcguucugcauuucggugau

SEQ ID NO: 2 - RNA, 40 nt, Artificial Sequence
  RNA oligonucleotide
aaacucaucgguagccuuccugcggucagucuauuaggac

SEQ ID NO: 3 - RNA, 40 nt, Artificial Sequence
  RNA oligonucleotide
agcgacugacgaucuugaguaaaccgcucauccacguagu

SEQ ID NO: 4 - RNA, 40 nt, Artificial Sequence
  RNA oligonucleotide
ucaccagcgcucuacgaaccccgcauucccaguugcuaca

SEQ ID NO: 5 - RNA, 15 nt, Artificial Sequence
  RNA oligonucleotide
gggaggacgaugcgg

SEQ ID NO: 6 - RNA, 25 nt, Artificial Sequence
  RNA oligonucleotide
cagacgacucgcugaggauccgaga

SEQ ID NO: 7 - RNA, 80 nt, Artificial Sequence
  RNA oligonucleotide
gggaggacgaugcggccgauuagaacugggcugcgaucggagauccucuagguuucagacgacucgcugaggauccgaga

SEQ ID NO: 8 - RNA, 80 nt, Artificial Sequence
  RNA oligonucleotide
gggaggacgaugcgggccgagggauucgguguagacucugcacaguccugaaaagcagacgacucgcugaggauccgaga

SEQ ID NO: 9 - RNA, 80 nt, Artificial Sequence
  RNA oligonucleotide
gggaggacgaugcggccgauuagaacugggcugaggcguucugcauuucggugaucagacgacucgcugaggauccgaga

SEQ ID NO: 10 - RNA, 80 nt, Artificial Sequence
  RNA oligonucleotide
gggaggacgaugcggccgauuagaacugggcuguuccgacucugaauccggugaucagacgacucgcugaggauccgaga

SEQ ID NO: 11 - RNA, 80 nt, Artificial Sequence
  RNA oligonucleotide
gggaggacgaugcgguuggugaagggaggucagcauaucuucccgcgggaagcgacggacgacucgcugaggauccgaga

SEQ ID NO: 12 - RNA, 80 nt, Artificial Sequence
  RNA oligonucleotide
gggaggacgaugcggauccacggguaagggugagggacgacaucaaggcgagauucagacgacucgcugaggauccgaga

SEQ ID NO: 13 - RNA, 80 nt, Artificial Sequence
  RNA oligonucleotide
gggaggacgaugcgguacgauuagaacuggugccgaacagcgcucguugaagacacagacgacucgcugaggauccgaga

SEQ ID NO: 14 - RNA, 80 nt, Artificial Sequence
  RNA oligonucleotide
gggaggacgaugcggaggaaguaggguucguccauugggcgaguggccuguguuacagacgacucgcugaggauccgaga

SEQ ID NO: 15 - RNA, 80 nt, Artificial Sequence
  RNA oligonucleotide
gggaggacgaugcggcacgguauaguggaguggguaggcaucgcucgacgagugacagacgacucgcugaggauccgaga

SEQ ID NO: 16 - RNA, 80 nt, Artificial Sequence
  RNA oligonucleotide
gggaggacgaugcgggaguagggagguaaucgccaccccaacgcggagacagcgacagacgacucgcugaggauccgaga

SEQ ID NO: 17 - RNA, 80 nt, Artificial Sequence
  RNA oligonucleotide
gggaggacgaugcggucgauagggggacggaccgcgcuggaaacucaacguagcacagacgacucgcugaggauccgaga

SEQ ID NO: 18 - RNA, 80 nt, Artificial Sequence
  RNA oligonucleotide
gggaggacgaugcggcacugaugggagugggaucagugucgagcgguaucugcagcagacgacucgcugaggauccgaga

SEQ ID NO: 19 - RNA, 80 nt, Artificial Sequence
  RNA oligonucleotide
gggaggacgaugcgggaguagggagguaaucgucaccccaacgcggagacagcgacagacgacucgcugaggauccgaga

SEQ ID NO: 20 - RNA, 79 nt, Artificial Sequence
  RNA oligonucleotide
gggaggacgaugcggaagcauacaguuuagugugcuagggugggacucagugaucagacgacucgcugaggauccgaga

SEQ ID NO: 21 - RNA, 80 nt, Artificial Sequence
  RNA oligonucleotide
gggaggacgaugcgguccuacuuuccccaauuuguaacagcucuccgcacagcagcagacgacucgcugaggauccgaga

SEQ ID NO: 22 - RNA, 80 nt, Artificial Sequence
  RNA oligonucleotide
gggaggacgaugcggcgguguagggaucgucagucucggaaugaccucacagaagcagacgacucgcugaggauccgaga

SEQ ID NO: 23 - RNA, 80 nt, Artificial Sequence
  RNA oligonucleotide
gggaggacgaugcggccagcaggaggaugggugccgcacucggauauucacgugucagacgacucgcugaggauccgaga

SEQ ID NO: 24 - RNA, 80 nt, Artificial Sequence
  RNA oligonucleotide
gggaggacgaugcgggacuaagcacaacucaacuagaacgaaccuauuccaucaucagacgacucgcugaggauccgaga

SEQ ID NO: 25 - RNA, 80 nt, Artificial Sequence
  RNA oligonucleotide
gggaggacgaugcggaacggaggaguguccucucagcugacagucgugcauacuacagacgacucgcugaggauccgaga

SEQ ID NO: 26 - RNA, 80 nt, Artificial Sequence
  RNA oligonucleotide
gggaggacgaugcggaacucgauccaucaucgugacugcguacgugucaacuaagcagacgacucgcugaggauccgaga

SEQ ID NO: 27 - RNA, 80 nt, Artificial Sequence
  RNA oligonucleotide
gggaggacgaugcgggacggucauagagccggccgacauuagagccgggaauccacagacgacucgcugaggauccgaga

SEQ ID NO: 28 - DNA, 80 bp, Artificial Sequence
  RNA/DNA oligonucleotide
gggaggacgaugcgguguagggagguaaucuucuugccaaucuugguuacucgugtagacgacucgcugaggauccgaga

SEQ ID NO: 29 - RNA, 80 nt, Artificial Sequence
  RNA oligonucleotide
gggaggacgaugcgguggagaggggaaucguccugcgcacucugucuccugagagcagacgacucgcugaggauccgaga

SEQ ID NO: 30 - RNA, 80 nt, Artificial Sequence
  RNA oligonucleotide
gggaggacgaugcgguguauccgccaguaugauuaacaucuauaagucccuaugucagacgacucgcugaggauccgaga

SEQ ID NO: 31 - RNA, 80 nt, Artificial Sequence
  RNA oligonucleotide
gggaggacgaugcggcuaaccuuguuagggccccauacagcaucgagugacggaucagacgacucgcugaggauccgaga

SEQ ID NO: 32 - RNA, 80 nt, Artificial Sequence
  RNA oligonucleotide
gggaggacgaugcggugcacaggaggugguacacugcgcucgauucaucagcgcacagacgacucgcugaggauccgaga

SEQ ID NO: 33 - RNA, 80 nt, Artificial Sequence
  RNA oligonucleotide
gggaggacgaugcggcaugugagggaggagguccgcgucauaaacuccaggaccacagacgacucgcugaggauccgaga

SEQ ID NO: 34 - RNA, 80 nt, Artificial Sequence
  RNA oligonucleotide
gggaggacgaugcggaagcaauagcucgccguacaguuguccugccguucguguucagacgacucgcugaggauccgaga

SEQ ID NO: 35 - RNA, 80 nt, Artificial Sequence
  RNA oligonucleotide
gggaggacgaugcgggaguagggagguaagcagcggacuaacgagauucggugagcagacgacucgcugaggauccgaga

SEQ ID NO: 36 - RNA, 80 nt, Artificial Sequence
  RNA oligonucleotide
gggaggacgaugcggcgagcaaccggaacucggcuauuaugaccagccaacuuaacagacgacucgcugaggauccgaga

SEQ ID NO: 37 - RNA, 80 nt, Artificial Sequence
  RNA oligonucleotide
gggaggacgaugcggcgagcaaccugaacucggcuauuaggaccagccaacuuaacagacgacucgcugaggauccgaga

SEQ ID NO: 38 - RNA, 80 nt, Artificial Sequence
  RNA oligonucleotide
gggaggacgaugcgggaucggaaccagcgugacgaagcgcggaucaacuccggugcagacgacucgcugaggauccgaga

SEQ ID NO: 39 - RNA, 80 nt, Artificial Sequence
  RNA oligonucleotide
gggaggacgaugcgggaucggaaccagcgugacgaagcgcggaucaacuccggugcugacgacucgcugaggauccgaga

SEQ ID NO: 40 - RNA, 78 nt, Artificial Sequence
  RNA oligonucleotide
gggaggacgaugcggccgauuagaacugggucgcgcuguacccuagggaucgacagacgacucgcugaggauccgaga

SEQ ID NO: 41 - RNA, 80 nt, Artificial Sequence
  RNA oligonucleotide
gggaggacgaugcggagacccaagcgccacgucuggcaugugagggaggagguaccagacgacucgcugaggauccgaga

SEQ ID NO: 42 - RNA, 80 nt, Artificial Sequence
  RNA oligonucleotide
gggaggacgaugcggagagccaagcgccacgucuggcaugugaggggggagguaccagacgacucgcugaggauccgaga

SEQ ID NO: 43 - RNA, 80 nt, Artificial Sequence
  RNA oligonucleotide
gggaggacgaugcggaaacucaucgguagccuuccugcggucagucuauuaggaccagacgacucgcugaggauccgaga

SEQ ID NO: 44 - RNA, 80 nt, Artificial Sequence
  RNA oligonucleotide
gggaggacgaugcggcaauuaccucagccucccuagacacgucgucuauuaggaccagacgacucgcugaggauccgaga

SEQ ID NO: 45 - RNA, 80 nt, Artificial Sequence
  RNA oligonucleotide
gggaggacgaugcggcagucuuccgguaagcacggaggugaggggagcuuagcgucagacgacucgcugaggauccgaga

SEQ ID NO: 46 - RNA, 80 nt, Artificial Sequence
  RNA oligonucleotide
gggaggacgaugcggauaugccaugggucgcucgagugaggucgucuauuaggaccagacgacucgcugaggauccgaga

SEQ ID NO: 47 - RNA, 80 nt, Artificial Sequence
  RNA oligonucleotide
gggaggacgaugcggagagccaagcgccacgucuggcaugugagggaggagguaccagacgacucgcugaggauccgaga

SEQ ID NO: 48 - RNA, 80 nt, Artificial Sequence
  RNA oligonucleotide
gggaggacgaugcggauuggcgcuauuaggaccagcuccguccgcaacuggucccgagacgacucgcugaggauccgaga

SEQ ID NO: 49 - RNA, 80 nt, Artificial Sequence
  RNA oligonucleotide
gggaggacgaugcgggaacagacccauggcaaucucgcgacgucuucggccgcugcagacgacucgcugaggauccgaga

SEQ ID NO: 50 - RNA, 79 nt, Artificial Sequence
  RNA oligonucleotide
gggaggacgaugcgguacaacagguucauacggcgcguuguuccuuggcugacgcagacgacucgcugaggauccgaga

SEQ ID NO: 51 - RNA, 80 nt, Artificial Sequence
  RNA oligonucleotide
gggaggacgaugcggcacuauuaggaccagugccuguugucucgauaagcuccgccagacgacucgcugaggauccgaga

SEQ ID NO: 52 - RNA, 80 nt, Artificial Sequence
  RNA oligonucleotide
gggaggacgaugcggauuggcgcuauuaggaccagcuccguccgcaacugaucccgagacgacucgcugaggauccgaga

SEQ ID NO: 53 - RNA, 80 nt, Artificial Sequence
  RNA oligonucleotide
gggaggacgaugcggcuauuaggaccagccguguagaauucguagcgaugugacgcagacgacucgcugaggauccgaga

SEQ ID NO: 54 - RNA, 80 nt, Artificial Sequence
  RNA oligonucleotide
gggaggacgaugcgguucgcgcuauuaggaccagugcgaacguggguauacaugucagacgacucgcugaggauccgaga

SEQ ID NO: 55 - RNA, 80 nt, Artificial Sequence
  RNA oligonucleotide
gggaggacgaugcggaacacacgggacgagccuggcgguugucgucuauuaggaccagacgacucgcugaggauccgaga

SEQ ID NO: 56 - RNA, 81 nt, Artificial Sequence
  RNA oligonucleotide
gggaggacgaugcggguccaugcuuuaaacugcaauuucccguuuacacgggcugucagacgacucgcugaggauccgaga

SEQ ID NO: 57 - RNA, 80 nt, Artificial Sequence
  RNA oligonucleotide
gggaggacgaugcggaccaccgaaucacgaggugcgagacauugguuccccgccgcagacgacucgcugaggauccgaga

SEQ ID NO: 58 - RNA, 80 nt, Artificial Sequence
  RNA oligonucleotide
gggaggacgaugcgggggacaauaguccacgacuacaugucggugcgucggaggucagacgacucgcugaggauccgaga

SEQ ID NO: 59 - RNA, 80 nt, Artificial Sequence
  RNA oligonucleotide
gggaggacgaugcggcuauuaggaccagcugccaauguuaagucuaccccagcagcagacgacucgcugaggauccgaga

SEQ ID NO: 60 - RNA, 80 nt, Artificial Sequence
  RNA oligonucleotide
gggaggacgaugcggcuuacguauggucacggaggugugggggaacauacagcagcagacgacucgcugaggauccgaga

SEQ ID NO: 61 - RNA, 80 nt, Artificial Sequence
  RNA oligonucleotide
gggaggacgaugcgguuggugaccuauucaggcguaggcauauaaacuacgaggccagacgacucgcugaggauccgaga

SEQ ID NO: 62 - RNA, 80 nt, Artificial Sequence
  RNA oligonucleotide
gggaggacgaugcggcuauuaggaccagcugccaauguuaagucuaccccagcggcagacgacucgcugaggauccgaga

SEQ ID NO: 63 - RNA, 80 nt, Artificial Sequence
  RNA oligonucleotide
gggaggacgaugcgggcacgacacgccgauuagaacugggcgaucuuggucgagccagacgacucgcugaggauccgaga

SEQ ID NO: 64 - RNA, 80 nt, Artificial Sequence
  RNA oligonucleotide
gggaggacgaugcggcgauacgaccgcaugaguauaccgucgugcuucccggcugcagacgacucgcugaggauccgaga

SEQ ID NO: 65 - RNA, 80 nt, Artificial Sequence
  RNA oligonucleotide
gggaggacgaugcggauuggcgcuauuaggaccagcuccguccgcaaccgguccccagacgacucgcugaggauccgaga

SEQ ID NO: 66 - RNA, 80 nt, Artificial Sequence
  RNA oligonucleotide
gggaggacgaugcggauuggcgcuauuaggaccagcuccguccgcaacugguccccagacgacucgcugaggauccgaga